(19) United States
(12) Patent Application Publication
(10) Pub. No.: US 2016/0000341 A1
(43) Pub. Date: Jan. 7, 2016

(54) INTRAVASCULAR PRESSURE DROP DERIVED ARTERIAL STIFFNESS AND REDUCTION OF COMMON MODE PRESSURE EFFECT

(71) Applicant: RAMOT AT TEL-AVIV UNIVERSITY LTD., Tel Aviv (IL)

(72) Inventors: Oren ROTMAN, Holon (IL); Shmuel EINAV, Herzlia (IL)

(21) Appl. No.: 14/768,461

(22) PCT Filed: Feb. 18, 2014
(86) PCT No.: PCT/IL2014/050181

§ 371 Date: Aug. 18, 2015

Related U.S. Application Data
(60) Provisional application No. 61/765,835, filed on Feb. 18, 2013.

(51) Int. Cl. A61B 5/0215; A61B 5/00; A61B 18/14

(52) U.S. Cl. CPC A61B 5/02158; A61B 18/1492; A61B 5/7225; A61B 2018/00642

(57) ABSTRACT

In some embodiments, a method and/or device are disclosed for measuring a pressure drop over a portion of a body lumen. Optionally, the pressure drop for two different flow conditions may be used to find the stiffness of the lumen. In some embodiments the device may be calibrated, for example by correcting for distortion, for example the common mode pressure distortion. Pressure drop and or stiffness measures may be used to evaluate a treatment procedure and/or a stenosis.

### FIELD AND BACKGROUND OF THE INVENTION

The present invention, in some embodiments thereof, relates to intravascular catheters and measurements made with them, more particularly, but not exclusively, to measurements of pressure drop and arterial stiffness.

Cardiovascular disease has long been the leading cause of death in the western world: 26.8 million non-institutionalized adults with diagnosed heart disease in 2009 (Pleis, J. R., B. W. Ward, et al. (2010), “Summary health statistics for U.S. adults: National Health Interview Survey, 2009.” Vital Health Stat 10(249): 1-207) and approximately 616,000 heart disease related deaths at 2007 (Xu, J. K., Kenneth D; Murphy, Sherry L; Tejada-Vera, Betzaida (2010), “Deaths: Final data for 2007.” National Vital Statistics Reports 58(19)) in the US alone. The accepted standard tool of cardiologists for assessing stenoses severity in the coronary tree is coronary angiography, in which the coronary arteries lumen is visualized; a radio-opaque contrast agent is being injected during a catheterization procedure into the coronary arteries, and imaged by X-ray based technique. However, the information given from this technique is limited to projection of two-dimensional visualization image of the lumen only, providing limited functional data on the severity of a stenosis; does the visualized stenosis induce ischemia and should be treated? In addition, histopathological studies have demonstrated that angiographic evidence of stenosis is usually not detected until the cross-sectional area of plaque approaches 40% to 50% of the total cross-sectional area of the vessel (Tobis, J., B. Azarbal, et al. (2007), “Assessment of intermediate severity coronary lesions in the catheterization laboratory.” J Am Coll Cardiol 49(8): 839-848). These limitations of coronary angiography, and in addition significant intra- and inter-observer variability in assessment of stenoses, makes the management of intermediate coronary lesions (defined by a diameter stenosis of 40% to 70%) to be truly challenging for cardiologist.

Functional assessment of an atherosclerotic stenosis practically means assessment of ischemia induced by the stenosis, or how much does a single atherosclerotic stenosis reduces blood flow in comparison to the same artery theoretically without the stenosis. The coronary circulation can be viewed as a two-compartment model. The first compartment consists of large epicardial vessels (>400 microns), which have minimal resistance to blood flow and therefore the pressure drop along them in a normal condition is negligible; e.g. the left main coronary artery, left anterior descending (Promonet, C., D. Anglade, et al. (2000). “Time-dependent pressure distortion in a catheter-transducer system: correction by fast flush,” Anesthesiology 92(1): 208-218) and the left circumflex artery. The second compartment is the coronary microcirculation and consists of arteries smaller than 400 microns, or ‘resistive vessels’. Several indices of coronary physiology have been proposed for the estimation of to coronary circulatory function to guide clinical decision making, and will be described in the following sections.

### Coronary Flow Reserve (CFR)

Coronary flow reserve is defined as the ration of hyperemic blood flow (Q^{max}) to resting myocardial blood flow (Q^{rest}):

$$CFR = \frac{{Q}^{max}}{{Q}^{rest}} \qquad Eq . 1$$

The normal value for CFR is still not well defined and normal values differ from study to study. There is some consensus of opinion, however, suggesting that a value more than 4 should be considered as normal, which means that microvascular resistance can decrease by a factor of 4 (Hamilos, M., A. Peace, et al. (2010), “Fractional flow reserve: an indispensable diagnostic tool in the cardiac catheterisation laboratory,” Hellenic J Cardiol 51(2): 133-141). Since absolute myocardial flow is not easy to determine, surrogate markers of flow are commonly used, such as flow velocities assessed by Doppler wire, or mean transit time (Tₘₙ) assessed by pressure/temperature wire. Pressure based CFR was also suggested and was validated both experimentally and numerically (Shalman, E., C. Barak, et al. (2001). “Pressure-based simultaneous CFR and FFR measurements: understanding the physiology of a stenosed vessel.” Comput Biol Med 31(5): 353-363; Shalman, E., M. Rosenfeld, et al. (2002), “Numerical modeling of the flow in stenosed coronary artery. The relationship between main hemodynamic parameters,” Comput Biol Med 32(5): 329-344). Regardless of the method used to measure CFR, this technique has several limitations (Hamilos, Peace et al. 2010): (1) resting flow is highly variable; (2) hyperaemic flow is directly dependant on systemic blood pressure; (3) the hyperaemic and resting measurements are not performed simultaneously but successively; (4) CFR is not specific for an epicardial stenosis, as the CFR value depends on both epicardial vessels and microcirculation. When CFR is low, it is impossible to distinguish whether this value is related to an epicardial artery stenosis alone, microcirculatory dysfunction alone, or a combination of both. (5) CFR depends on pharmacologically-induced hyperaemia, usually by intravenous administration of adenosine, however in 10%-15% of patients, intracoronary adenosine induces submaximal hyperaemia only, and therefore CFR would be underestimated (Pijls, N. H., M. J. Kern, et al. (2000). “Practice and potential pitfalls of coronary pressure measurement.” Catheter Cardiovasc Intery 49(1): 1-16). Because of these limitations, CFR has only limited value in clinical decision making.

### Index of Microcirculatory Resistance (IMR)

The myocardial microcirculatory system, rather than the large conduit epicardial coronary arteries, is responsible for most of the resistance to coronary flow. A measure of the resistance of the former will give an indication of their function. Microcirculatory resistance (R) is derived by the pressure drop across the microcirculation divided by the flow (Fearon, W. F., L. B. Balsam, et al. (2003), “Novel index for invasively assessing the coronary microcirculation,” Circulation 107(25): 3129-3132):

$$R = \frac{( {P}_{d} - {P}_{v} )}{Q} \qquad Eq . 2$$

With maximal hyperemia, the pressure in the distal epicardial coronary artery (proximal to the microcirculation) can be assumed to be the pressure drop, given that the pressure distal to the microcirculation (central venous pressure) can be assumed to be zero:

$$R = \frac{{P}_{d}}{{Q}^{max}} \qquad Eq . 3$$

However, absolute coronary flow cannot be easily measured in the catheterization laboratory and, therefore, true microcirculatory resistance cannot be measured in the clinical setting. An index of microcirculatory resistance (IMR) has shown to correlate well with the true microcirculatory resistance, considering that mean transit time (Tₘₙ) is inversely proportional to coronary flow during hyperemia (Fearon, Balsam et al. 2003). Therefore, during maximal hyperemia:

IMR=P_{d}·Tₘₙ Eq.4

Unlike CFR measurements, IMR is associated with much lower variability and is not significantly affected by changes in hemodynamic conditions (Leung, D. Y. and M. Leung (2011), “Non-invasive/invasive imaging: significance and assessment of coronary microvascular dysfunction.” Heart 97(7): 587-595).
Fractional Flow Reserve indicator (FFR)

Fractional flow reserve is defined as the ratio of hyperemic flow in the stenotic artery (Q_{S}^{max}) to the flow in the same artery in the theoretic absence of the stenosis, meaning normal hyperemic myocardial flow (Q_{N}^{max}) (De Bruyne, B. and J. Sarma (2008), “Fractional flow reserve: a review: invasive imaging,” Heart 94(7): 949-959):

$$FFR = \frac{{Q}_{S}^{max}}{{Q}_{M}^{max}} \qquad Eq . 5$$

Since flow is the ratio of pressure (P) difference across the coronary system divided by its resistance (R), Q can be substituted as following:

$$FFR = \frac{( {P}_{d} - {P}_{v} ) {R}_{S}^{max}}{( {P}_{a} - {P}_{v} ) {R}_{N}^{max}} \qquad Eq . 6$$

When P_{d} and Pₐ are the mean coronary distal pressure and mean aortic pressure respectively, Pᵥ is the mean central venous pressure, and R_{S}^{max} and R_{N}^{max} are the hyperemic resistances of the stenotic and normal arteries respectively. Since measurements are obtained under maximal hyperemia, resistances are minimal and therefore equal, and thus they cancel out:

$$FFR = \frac{( {P}_{d} - {P}_{v} )}{( {P}_{a} - {P}_{v} )} \qquad Eq . 7$$

In addition, Pᵥ is negligible as compared to Pₐ or P_{d}, therefore:

$$FFR = \frac{{P}_{d}}{{P}_{a}} \qquad Eq . 8$$

Therefore in practice FFR uses pressure as a surrogate for flow, and represents the extent to which maximal myocardial blood flow is limited by the presence of an epicardial stenosis. If FFR is 0.60, it means that maximal myocardial blood flow to reaches only 60% of its normal value. FFR values of 0.75-0.80 have been established as threshold values that distinguish normal from abnormal levels for a given measurement; stenoses with an FFR<0.75 are considered as a cause of myocardial ischemia, whereas stenosis with an FFR>0.80 are considered to be ischemic ‘safe’ (De Bruyne and Sarma 2008). FFR takes into account the contribution of collaterals to myocardial perfusion during hyperemia, and in addition is not influenced by physiological variations in blood pressure and heart rate.

FFR is calculated from two simultaneous pressure measurements; aortic pressure by fluid filled catheter which is connected to a pressure gauge, and distal coronary pressure by pressure monitoring guidewires (catheter tipped pressure sensors).

As described previously, one of the limitations of FFR is its absolute dependency in pharmacologically-induced steady-state maximal hyperaemia. The current clinical standard for coronary hyperaemia is the intracoronary administration of adenosine, however in 10%-15% of patients, intracoronary adenosine induces submaximal hyperemia only and therefore FFR may be overestimated by up to 0.10 (Pijls, Kern et al. 2000).

FFR catheters basically use a catheter-tipped pressure transducer (a single gauge pressure transducer). Companies that commercialize such technology include (1) PressureWire™ Certus by Radi Medical Systems/St. Jude Medical™, (2) Volcano Corp., (3) Millar Instruments.

Arterial distensibility has long been one of the measures being investigated for assessing arterial stiffness as an indicator for the arterial health condition; e.g. correlation with stenosis severity, negative or positive plaque remodelling, and distinguishing white or yellow plaques. Normal arterial distensibility in large arteries is in the range of several percent diameter change; approximately 10% in the common carotid artery (Schmidt-Trucksass, A., D. Grathwohl, et al. (1999), “Structural, functional, and hemodynamic changes of the common carotid artery with age in male subjects,” Arterioscler Thromb Vasc Biol 19(4): 1091-1097; Mokhtari-Dizaji, M., M. Montazeri, et al. (2006), “Differentiation of mild and severe stenosis with motion to estimation in ultrasound images,” Ultrasound Med Biol 32(10): 1493-1498), and 4.5-6% in the large coronary arteries (Shimazu, T., M. Hori, et al. (1986), “Clinical assessment of elastic properties of large coronary arteries: pressure-diameter relationship and dynamic incremental elastic modulus.” Int J Cardiol 13(1): 27-45). Atherosclerosis was found to be associated with impaired distensibility in comparison to normal healthy arteries (van Popele, N. M., D. E. Grobbee, et al. (2001), “Association between arterial stiffness and atherosclerosis: the Rotterdam Study,” Stroke 32(2): 454-460), even in sites accompanying occult atherosclerosis, which cannot be detected by conventional angiography (Nakatani, S., M. Yamagishi, et al. (1995). “Assessment of coronary artery distensibility by intravascular ultrasound: Application of simultaneous measurements of luminal area and pressure,” Circulation 91(12): 2904-2910). Mokhtari-Dizaji et-al have found that relative diameter changes in CCA with mild and severe stenosis were decreased by 22% to 48%, respectively, compared with healthy carotid artery. The relative diameter change in the healthy, mild stenosis, and severe stenosis groups were 9.9±0.8%, 7.8±0.9%, and 5.2±0.5%, respectively. In addition, the stiffness indices were significantly different in the group of patients with severe stenosis compared with healthy and mild stenosis subjects (Mokhtari-Dizaji, Montazeri et al. 2006).

Konala et al, “Influence of Arterial Wall Compliance on the Pressure Drop across Arterial Wall Stenoses under Hyperemic Flow Condition,” MCB vol. 8, no. 1, p. 1-20 (2011), evaluates the influence in flow and pressure drop caused by variation in arterial-stenosis compliance for a wide range of stenosis severities. The flow and time-averaged pressure drop were evaluated for three different severities of stenosis and tested for limiting scenarios of compliant models. The Mooney-Rivlin model defined the non-linear material properties of the arterial wall and the plaque regions. The non-Newtonian Carreau model was used to model the blood flow viscosity. The fluid (blood)-structure (arterial wall) interaction equations were solved numerically using the finite element method. Irrespective of the stenosis severity, the compliant models produced a lower pressure drop than the rigid artery due to compliance of the plaque region, with a wide variation in pressure drop between different compliant models for significant (90% area occlusion) stenosis. These significant variations in pressure drop may lead to misinterpretation and misdiagnosis of the stenosis severity.

Konala et al, “Influence of arterial wall-stenosis compliance on the coronary diagnostic parameters,” Journal of Biomechanics 44 (2011), 842-847, evaluates the effect of arterial wall compliance, with limiting scenarios of stenosis severity, on functional diagnostic parameters. The diagnostic parameters considered include an established index, Fractional Flow Reserve indicator (FFR), and two recently developed parameters, Pressure Drop Coefficient (PDC), and Lesion Flow Coefficient (LFC). The study found that, with an increase in stenosis severity, FFR decreased whereas PDC and LFC increased. For fixed stenosis, CDP value decreased and LFC value increased with a decrease in plaque elasticity. The difference in diagnostic parameters with compliance at intermediate stenosis (78% to 83% area blockage) could lead to misinterpretation of the stenosis severity.

U.S. Pat. No. 4,901,731 to Millar describes an apparatus and method for sensing in vivo the fluid pressure differential between spaced locations in a biological fluid vessel using a single pressure transducer. The transducer has a deformable member mounted to a housing; a conduit extends within the housing with one end opening at a location spaced from the transducer and the other end opening adjoining the inner surface of the member. With the housing inserted in the biological fluid vessel, the outer surface of the deformable member is exposed to the fluid pressure adjacent the member, while the inner surface is exposed to the fluid pressure within the conduit. The deformable member flexes in response to the fluid pressure differential across the member, which is a direct measure of the fluid pressure differential between spaced-apart locations in the fluid-filled vessel. Strain gauges are mounted to the member to generate a signal indicative of the pressure differential, with electrical leads coupled to the strain gauges and received in a catheter threaded in the vessel. In a preferred embodiment, the transducer is mounted proximal to an angioplasty balloon and the conduit opens distal to the balloon. This arrangement can give a pressure differential across a lesion with the balloon positioned adjacent the lesion in the coronary arterial tree.

Gauge pressure measurements using fluid-filled catheters are widely used in medical practice for blood pressure measurements and known to distort pressure signals according to the catheter system transfer function (Shinozaki, T., Deane, R. S., Mazuzan, J. E., 1980, “The dynamic responses of liquid-filled catheter systems for direct measurements of blood pressure,” Anesthesiology 53, 498-504). The transfer function is affected by the measurement system components: the catheter diameter, catheter length, catheter material, the fluid viscosity, presence of air bubbles inside the extension tubing, and the pressure transducer characteristics (Hunziker, P., 1987, “Accuracy and dynamic response of disposable pressure transducer-tubing systems,” Can J Anaesth 34, 409-414). Under a good approximation, the fluid-filled catheter can be characterized as a second-order linear system, and the output signal can be then corrected using an inverse transfer function (Glantz, S. A., Tyberg, J. V., 1979, “Determination of frequency response from step response: application to fluid-filled catheters,” The American journal of physiology 236, H376-378; Lambermont, B., Gerard, P., Detry, O., Kolh, P., Potty, P., D'Orio, V., Marcelle, R., 1998, “Correction of pressure waveforms recorded by fluid-filled catheter recording systems: a new method using a transfer equation,” Acta Anaesthesiol Scand 42, 717-720).

### SUMMARY OF THE INVENTION

According to an aspect of some embodiments of the present invention there is provided a system for measuring stiffness of a body lumen comprising: a probe including two measuring locations; a pressure gauge, that generates a signal indicative of the differential pressure between the measurement locations; and a controller, adapted to compute a pressure drop between the measuring locations based on the signal of the pressure gauge, for each of at least two different flow conditions, and to find a relative stiffness of the body lumen from the pressure drops.

According to some embodiments of the invention, the system also includes a pressure-measuring catheter including: a fluid-filled first lumen and wherein a first of the measurement locations includes an opening from the first lumen to an outside surface of the pressure-measuring catheter and the pressure measuring catheter also includes a fluid-filled second lumen, and wherein a second of the measurement locations includes an opening from the second lumen to the outside surface of the pressure-measuring catheter, both the openings being inside the body lumen when the pressure-measuring catheter is inserted, the opening of the first lumen being more distal than the opening of the second lumen, and both the first lumen and the second lumen having proximal ends outside the body when the pressure-measuring catheter is inserted; and wherein the sensor measures a pressure differential at one or more of the proximal ends of the first and second lumens and the signal depends the measured pressure differential to between the first and second lumens.

According to some embodiments of the invention, the body lumen is a blood vessel and the pressure-measuring catheter is configured for insertion into the blood vessel, and the controller is adapted to find a pressure drop from the differential pressure signal for the at least two different flow conditions including at least two different phases of a cardiac cycle, and wherein the controller is adapted to find the relative stiffness from the pressure drops of the at least two different phases.

According to some embodiments of the invention, the system also comprises a flow sensor adapted to generate a signal indicative of flow in the body lumen, and wherein the controller is adapted to use the signal indicative of flow, obtained for the at least two flow conditions, to find an absolute stiffness of the body lumen from the pressure drops.

According to some embodiments of the invention, the system also comprises a sleeve that surrounds the pressure-measuring catheter, the pressure-measuring catheter being adapted to withdraw into and extend out of the sleeve when it is inserted into the body lumen, wherein the controller is adapted to discern a distortion of the signal generated by the pressure gauge when the pressure-measuring catheter is withdrawn into the sleeve.

According to some embodiments of the invention, the sleeve includes a delivery catheter.

According to some embodiments of the invention, the controller is further configured to calculate a correction for the distortion to the signal and to apply the correction for the distortion to the signal generated by the pressure gauge when the pressure-measuring catheter is exposed to the flow in the body lumen, the correction based on a result of the discerning.

According to some embodiments of the invention, the distortion includes a common mode pressure distortion.

According to some embodiments of the invention, a distance between the measuring locations is no more than 5 cm.

According to some embodiments of the invention, each opening of the first and second openings is substantially at a distal end of each respective the lumen of the catheter.

According to some embodiments of the invention, the system also comprises an interventional device for performing a treatment intervention on the body lumen and wherein the pressure-measuring catheter is used for verifying or monitoring the treatment intervention or both.

According to some embodiments of the invention, the interventional device includes an ablation device.

According to some embodiments of the invention, the controller is adapted to compute the pressure drop to an accuracy to within 0.1 mmHg.

According to some embodiments of the invention, the pressure gauge includes a differential pressure gauge.

According to an aspect of some embodiments of the present invention there is provided a method of measuring stiffness of a blood vessel in a subject, the method comprising: measuring a pressure drop across substantially the same portion of the blood vessel, for each of at least two different flow conditions; and analyzing the measured pressure drops to determine a relative stiffness of the blood vessel.

According to some embodiments of the invention, the portion has a length of less than 5 cm.

According to some embodiments of the invention, the measuring is to an accuracy to within 0.1 mmHg.

According to some embodiments of the invention, the method also comprises measuring the pressure drop for each of at least two flow conditions across at least another portion along the blood vessel, and analyzing the measured pressure drops to determine at least a relative stiffness comprises comparing the pressure drops measured across the same portion and the at least another portion to determine the relative stiffness.

According to some embodiments of the invention, the same portion and the at least another portion have the same length.

According to some embodiments of the invention, measuring the pressure drop across substantially the same portion and the another portion of a blood vessel comprises measuring the pressure drop using a sensor mounted on a catheter inserted into the blood vessel, and moving the catheter along the blood vessel to successively measure the pressure drop at substantially the same portion and at the another portion with the sensor.

According to some embodiments, the method also comprises evaluating a reduction in blood flow caused by the resistance in the same portion based on the pressure drop.

According to some embodiments of the invention, each of the at least two different flow conditions includes a different phase of the cardiac cycle.

According to some embodiments of the invention, the method also comprises measuring a blood flow rate in the blood vessel at the two different phases, and analyzing the measured pressure drops comprises also using the measured blood flow rates and determining an absolute stiffness.

According to some embodiments of the invention, measuring the pressure drop comprises: measuring with a multi-lumen catheter comprising two fluid-filled lumens with their distal ends exposed to the blood pressure at two different locations along the blood vessel, when the catheter is inserted in the blood vessel, and connected at their proximal ends to a pressure sensor located outside the body of the subject; and correcting the results of the measurement for a common mode pressure distortion.

According to some embodiments of the invention, measuring the pressure drop comprises measuring with a pressure sensor mounted on a catheter and located inside the blood vessel when the measurement is made.

According to some embodiments of the invention, the method also comprises using the determined stiffness to locate or evaluate one or more of a stenosis, a sclerotic lesion, and vulnerable plaque.

According to some embodiments of the invention, the method also comprises assessing an interventional treatment based on the determined stiffness.

According to some embodiments of the invention, the assessing an interventional treatment includes, verifying the treatment or monitoring progress of the treatment, or both.

According to some embodiments of the invention, the interventional treatment is of a renal denervation.

According to an aspect of some embodiments of the present invention there is provided a system for measuring pressure in a body lumen comprising: a fluid-filled first to lumen, a distal end of the first lumen opening on an outside surface of the catheter; and a fluid-filled second lumen, a distal end of the second lumen opening on the outside surface of the catheter, both the openings being inside a body lumen when the catheter is inserted, the opening of the first lumen being more distal than the opening of the second lumen, and both lumens having proximal ends outside the body when the catheter is inserted; a pressure gauge, adapted for connecting to the proximal ends of the first and second lumens, that generates a signal indicative of the differential pressure between the first and second lumens; and a controller, adapted to compute a pressure drop between the distal openings in the first and second lumen based on the signal of the pressure gauge.

According to some embodiments, the system also comprises a sleeve that surrounds the pressure-measuring catheter, the pressure-measuring catheter being adapted to withdraw into and extend out of the sleeve when it is inserted into the body lumen, and wherein the controller is adapted to discern a distortion of a differential pressure signal generated when the pressure-measuring catheter is withdrawn into the sleeve.

According to some embodiments of the invention, the sleeve includes a delivery catheter.

According to some embodiments of the invention, the controller is further configured to calculate and apply a correction for the distortion based on a result of the discerning to a differential pressure signal generated when the pressure-measuring catheter is extended out of the sleeve.

According to some embodiments of the invention, controller is further configured to output from the applied correction a differential pressure accurate to within 0.1 mmHg.

According to some embodiments of the invention, the controller is adapted to compute a Fractional Flow Reserve indicator.

According to some embodiments of the invention, the distortion is a common mode pressure distortion.

According to some embodiments of the invention, the pressure gauge includes a differential pressure gauge.

According to some embodiments of the invention, the system also comprises a distance indicating element that indicates how far the pressure-measuring catheter is inserted into the blood vessel, wherein the controller is adapted to record the pressure drop found at a plurality of different locations along the blood vessel by moving the pressure-measuring catheter different distances into the blood vessel, to record the location of each recorded pressure drop, and to use the pressure drops at the different locations to find the relative stiffness of the blood vessel at the different locations.

According to an aspect of some embodiments of the present invention there is provided a method of correcting a distortion of differential pressure between two sensing locations, the method comprising: inserting the probe into a first region of fluid with a time-varying gauge pressure but negligible pressure drop between the sensing locations; measuring an indicator of the time-varying gauge pressure in the first region; sensing the pressure drop under the time-varying gauge pressure in the first region, and finding a restoration function of the indicator of time-varying gauge pressure in the first region for an output signal of the sensing in the first region.

According to some embodiments of the invention, each the sensing locations includes a distal opening to a separate respective fluid filled lumen of a multilumen catheter and wherein the sensing is of a differential pressure of the separate respective lumens at a proximal end of the separate respective lumens and wherein the restoration function corrects a common mode pressure distortion of the separate respective lumens.

According to some embodiments of the invention, the method also comprises: inserting the catheter into a second region of fluid with a pressure drop between the sensing locations; measuring the indicator of time-varying gauge pressure in the second region; sensing the pressure drop in the second region; and transforming an output signal of the sensing in the second region with the restoration function and a function of the measured indicator of time-varying gauge pressure to obtain a corrected pressure drop in the second region.

According to some embodiments of the invention, the corrected pressure that is accurate to within 0.05 mmHg.

According to some embodiments of the invention, the restoration function transforms the indicator of gauge pressure to a linear combination of a finite number of terms selected from at least the one of the indicator, a first order time derivative of the to indicator, a higher order derivative of the indicator, a linear function of the gauge pressure, a derivative of the liner function of the gauge pressure and a higher order derivative of the linear function of the gauge pressure.

According to some embodiments of the invention, the second region is an interior of the body lumen, and the first region is an interior of a sleeve.

According to some embodiments of the invention, the second region is an interior of the body lumen, and the first region is a region with negligible flow.

According to some embodiments of the invention, the body lumen is a blood vessel.

According to some embodiments of the invention, the sensing locations are separated, along the body lumen, by a distance that is less than 5 cm, when the multi-lumen catheter is inserted into the blood vessel.

According to some embodiments of the invention, method also includes using the corrected pressure drop, for at least two different flow conditions, to find a stiffness of the body lumen.

According to some embodiments of the invention, method also includes the two different flow conditions include at least a systole and diastole phases of the cardiac cycle.

According to an aspect of some embodiments of the present invention there is provided a compound device for measuring a pressure drop in a body lumen in-vivo comprising: a probe of a pressure drop between two sensing locations; a sleeve that surrounds the pressure-measuring probe when it is inserted into the blood vessel, the pressure-measuring catheter being adapted to withdraw into and extend out of the sleeve; a sensor generate a signal indicative the pressure drop between the sensing locations; a controller adapted to discern a distortion of the signal generated when the probe is surrounded by the sleeve.

According to some embodiments of the invention, the sleeve includes a delivery catheter.

According to some embodiments of the invention, the controller is further adapted to compute a restoration function of a differential pressure signal based on the discerned distortion, and to obtain a corrected pressure drop by applying the restoration to the signal generated by the sensor when the probe is extending out of the sleeve.

According to some embodiments of the invention, the probe and sleeve are configured for insertion into a blood vessel for measuring the pressure drop during least two different phases of a cardiac cycle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the to drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D are schematic side view, side cross-sectional view, and axial cross-sectional views of a catheter, with two lumens for pressure drop measurements, according to two exemplary embodiments of the invention;

FIGS. 2A-D are a schematic side view FIG. 2A, side cross-sectional view FIG. 2B, and axial cross-sectional views FIG. 2C,D of a catheter having a third lumen according to two exemplary embodiments of the invention;

FIGS. 3A, B are a schematic side view of a multilumen catheter withdrawn into FIG. 3A and extended out FIG. 3B of a delivery catheter, according to an exemplary embodiment of the invention;

FIGS. 4A,B are schematic views of a systems for measuring pressure drop in an artery and using the pressure drop to estimate stiffness of the artery, according to embodiments of the invention;

FIG. 5 is a flowchart, showing a method of measuring pressure drop and estimating stiffness of the artery, according to an exemplary embodiment of the invention;

FIG. 6 is a flowchart showing a method of measuring pressure drop and estimating stiffness of the artery, according to some embodiments of the invention;

FIG. 7 is a schematic view illustrating a method to estimate stiffness of a renal artery according to some embodiments of the invention;

FIG. 8 is a schematic view showing of a method to locate and/or estimate stenoses at different locations along a left main coronary artery according to an embodiment of the current invention;

FIG. 9 is a block diagram of a system to measure a pressure differential according to some embodiments of the current invention;

FIG. 10 is a flow chart illustration of a method to measure a pressure differential according to some embodiments of the current invention;

FIG. 11 is a plot of in vitro test data showing the raw and corrected data for pressure drop as a function of time, for two different cases, using a restoration function, according to an exemplary embodiment of the invention;

FIG. 12 is graph of pressure drop measured in an artery undergoing ablation according to an exemplary embodiment of the invention; and

FIG. 13 is a graph of pressure drop measured by a differential pressure probe pulled along a lumen with variable distensibility according to an exemplary embodiment of the invention.

### DESCRIPTION OF SPECIFIC EMBODIMENTS OF THE INVENTION

The present invention, in some embodiments thereof, relates to intravascular catheters and measurements made with them, more particularly, but not exclusively, to measurements of pressure drop and arterial stiffness.

As used herein, arterial stiffness means the ratio of the change in pressure to the change in volume of an artery, for small changes. Distensibility and/or compliance of an artery is proportional to the ratio of the relative change in volume of the artery to the change in pressure, for small changes. Distensibility is also sometimes used loosely herein to refer to the relative change in volume of an artery produced by the peak-to-peak difference in pressure between systole and diastole. Since the volume and peak-to-peak pressure of an artery are generally known, the stiffness just depends on the inverse of the distensibility and/or compliance, and expressions such as finding, measuring or estimating the stiffness of an artery, as used herein, have the same meaning as finding, measuring or estimating the distensibility and/or compliance. Transforming between compliance, distensibility and/or stiffness may imply knowledge of the diameter of the artery. In some embodiments of the current invention diameter along a body lumen is measured (for example using x-rays and/or x-ray visible dies to mark and/or discern a body lumen). The diameter measurement may in some embodiments be used to compute and/or adjust relative and/or absolute values of compliance, stiffness, and/or distensibilty.

An aspect of some embodiments of the invention concerns finding the stiffness of a body lumen, for example a blood vessel, for example an artery, from pressure drop measurements made by an intravascular catheter inserted into the blood vessel. Optionally, the pressure drop is measured over a relatively short distance, for example between 5 and 10 cm and/or between 3 and 5 cm, and/or between 2 and 3 cm, and/or between 1 and 2 cm, and/or less than 1 cm, along the artery. Optionally, the pressure drop is measured at more than one phase of the cardiac cycle, for example at the systole and at the diastole, and the stiffness of the artery wall is found by comparing the pressure drop at the systole and the diastole. The pressure drops may optionally be used to find the change in diameter of the artery over changes in flow conditions, for example between systole and diastole. The change in diameter and/or the change in blood pressure between systole and diastole may be used to find the stiffness. As used herein the term flow conditions refers to a flow having a set of characteristic parameters. Changes in flow conditions may in some cases include changes in a flow regime. A different flow condition is a flow differing in at least one of those parameters. For example two different flow conditions of an incompressible fluid in a single section of a stiff tube may have the same volumetric flow rate and the same pressure drop but differ in the background pressure (for example in one regime the pressure drops from 1 ATM to 0 ATM while in the other flow conditions pressure drops from 2 ATM to 1 ATM). For a distensible tube, for example changing the flow conditions by raising the background pressure may change the diameter of the tube resulting in a different pressure drop for the same flow rate.

In some embodiments of the current invention, knowledge of flow parameters for different flow conditions in a single tube section (for example for substantially the same portion of a body lumen) may be used to determine the stiffness of the tube section (and/or portion of the body lumen). Alternatively or additionally, knowledge of flow parameters in different tube sections (and/or different portions of a body lumen) may allow determination of relative properties of the different sections (and/or portions). For example, measurement of pressure drop for two flow conditions may include measuring pressure drop over substantially the same portion of a blood vessel at two different phases of a cardiac cycle.

It should be understood that, in the description herein, any procedure that involves measuring, calculating, comparing, finding values, and similar actions is optionally done automatically by a controller, for example a programmable computer, a dedicated electronic circuit, or similar device. In the case where the controller is a to programmable computer, “adapted” and/or “configured” as used herein includes “programmed.” Alternatively, one or more such procedures are done manually by a user.

In some embodiments of the invention, the blood flow rate is measured by some kind of sensor, and is used when finding the artery diameter from the pressure drop. In other embodiments of the invention, the blood flow is not measured, but is assumed to be the substantially the same everywhere along the artery, if there is no major branching along the portion of the vessel considered, and a relative stiffness of the wall is found, at different points along the artery, from measurements of pressure drop made at each of the different points, even if the absolute stiffness is not found. The relative stiffness is used, for example, to identify locations that may have sclerotic lesions or vulnerable plaque, and to evaluate their severity and the need for intervention. A relative stiffness that is more than 20% higher or lower than the average stiffness along that artery, or more than 30% higher or lower, or more than 50% higher or lower, or more than a factor of 2 higher or lower, may point to a clinically significant pathology at that location. Higher stiffness in one location, for example distensibility less than 5%, or less than 70% of the average distensibility for that artery, may indicate an atheroma. A stenosis with lower stiffness, for example distensibility greater than 10%, or greater than the average distensibility for non-stenotic parts of the artery, may indicate vulnerable “active” plaque that is more likely to rupture or grow in the future. Estimates of relative stiffness may be used together with estimates of the reduction in blood flow caused by a stenosis, based on measurements of the pressure drop across the stenosis. Such a combination of functional data (reduction in blood flow) and mechanical data (change in stiffness) may be more useful than either one alone, in evaluating lesions, plaque and stenoses in arteries.

Optionally, the location of the catheter is found, each time the pressure drop is measured, by using a medical imaging system that shows the location of at least a portion of the catheter in the body, for example a fluoroscopic imaging system that shows the location of a radio-opaque marker on the catheter. Such an imaging system might be in use in any case if the catheter is also being used to inject a contrast agent for angiography. Alternatively, the location of the catheter is found from one or more markers on the catheter, visible outside the body, that indicate how far into the body the catheter has advanced, and make it possible to find, at least within about 1 cm, where along the artery the pressure drop was measured.

In some embodiments of the invention, the pressure drop is measured between two sensing locations. For example, the sensing locations include two pressure sensors, and/or two input zones to differential pressure sensor, mounted directly on the catheter, inside the body lumen. Alternatively or additional, the catheter may include two fluid-filled lumens. Each lumen may be in communication with a sensing location. For example, a sensing location may include a distal opening to the lumen. The distal opening of each lumen may be in a different position along the length of the catheter inside the artery. A differential pressure gauge may optionally include a sensor located outside the body and/or at the proximal end of the two lumens. The differential pressure gauge may optionally include a single sensor whose output signal is dependent on the pressure difference between the lumens. Alternatively or additionally, a pressure difference may be measured by two pressure sensors, each measuring pressure with respect to a reference pressure. For example the reference pressure may be atmospheric pressure and the measured pressure in each lumen may be gauge pressure. The differential pressure between the lumens may be computed by subtracting the gauge pressure in one lumen from the gauge pressure measured in the other lumen.

In some embodiments a pressure gauge may be calibrated. Optionally calibration may be conducted in-vivo (while the pressure measuring probe is located inside a living subject). Optionally, calibration may include removing a distortion from the signal output of a pressure sensor. For example, the pressure drop measured between two lumens may be corrected for a common mode pressure distortion, which results from the two long thin lumens having different transfer functions. In some cases, the common mode pressure distortion can be greater than the pressure drop, which may be quite small for example when the sensing locations are not too far apart. For example the sensing locations may be less than 5 cm apart and/or less than 3 cm apart and/or less than 2 cm apart. In some cases it may not be possible to measure the pressure drop accurately without correcting for the relatively large distortion.

An aspect of some embodiments of the invention concerns correcting a pressure drop, measured by an intravascular catheter, for a common mode pressure distortion. Making the correction comprises discerning the common mode pressure distortion, using to a pressure gauge, while measuring an indicator of the gauge pressure, both as a function of time. As used herein, when describing measurements inside an artery, “gauge pressure,” or “blood pressure,” means the blood pressure at a given location inside the artery as a function of time over one of more cardiac cycles. This is in contrast to “pressure drop,” which means the difference in gauge pressure between two points along the artery. Since the pressure drop is generally very small compared to the gauge pressure, even along the whole length of an artery from the aorta to the beginning of the microvasculature, it is generally not necessary, for purposes of this description, to specify the location at which gauge pressure is measured. The small pressure drop per length along an artery, on the other hand, can change significantly at different locations along the artery. For example, a pressure drop measurement may be associated with a portion of the artery at a particular location. Most of the drop in pressure in the circulatory system occurs in the microvasculature, defined herein as blood vessels smaller than 400 micrometers in diameter, and the pressure measurements described herein are made in much larger arteries.

In some embodiments of the invention, a pressure distortion is discerned by measuring the pressure drop in a location where there is negligible pressure drop between the sensing locations. For example a common mode pressure drop may be measured using a pressure gauge outside the body. The sensing locations optionally include openings of the two lumens. For example, a negligible pressure drop may occur when there is no substantial blood flowing along the catheter between the sensing locations. For example, a pressure drop may be negligible when it is between 10% and 30% of an intended measurement accuracy and/or a pressure drop may be negligible when it is between 1% and 10% of an intended measurement accuracy and/or a pressure drop may be negligible when it is less than 1% of an intended measurement accuracy. For example, when the measurement is to be accurate 0.1 mm Hg, 10% of the intended measurement accuracy would be 0.01 mm Hg.

In some embodiments, the multi-lumen catheter is inserted into the body inside a sleeve (for example a delivery catheter) that surrounds the multilumen catheter, and keeps it away from the blood flow, but still exposes the measuring locations of the probe to the pressure inside the body lumen. In the absence of blood flow along axis of the multi-lumen catheter, the pressure drop may be negligible between measuring locations to located along the axis of the catheter, i.e. the gauge pressure will be the same at both measuring locations. In some embodiments, each measuring location may include an opening to a respective lumen of the multilumen catheter. In these circumstances, the apparent pressure difference between the two lumens of the multilumen catheter, may be due entirely to the common mode pressure effect. In some embodiments, the differential pressure measured at their proximal ends outside the body by a differential pressure gauge. As used herein, the common mode pressure effect, or common mode pressure distortion, or common mode pressure error, means the difference in pressure measured by the differential pressure gauge, due to the differences in transfer function of the two catheter lumens, and/or any interaction between the two catheter lumens, when the transfer function acts on the time-varying gauge pressure present at the openings at the distal end of the two lumens.

Attempts of the inventors to measure pressure drops along a fluid flow in a cylindrical tube using a differential pressure transducer and double-lumen fluid-filled catheter have revealed that, in some of the cases examined, the most significant effect on the distortion of the output signal appears to be the common mode pressure effect (CMP). CMP may not be directly measured by differential pressure sensors, but it can superimpose any dynamics on the differential measurement, especially for very low differential pressure sensors. CMP increases due to physical differences between the negative and positive channels connected to the differential sensor. A physical difference between the channels may result in a different time delay between the channels, and even a difference on the scale of milliseconds can have a major effect on the differential measurement. It is exceedingly difficult to avoid the CMP effect. For example CMP may be significantly affected by the presence of air bubbles inside the tubing. To reduce the CMP effect, special care may be taken to remove air bubbles from the working fluid. However, even if the fluid is depressurized it may still not be enough. Another factor influencing the CMP is the diameter of the pressure line, or catheter lumen; small physical difference between the channels (e.g. a small kink in the wall or a micro-bubble of air in one of the channels) may be relatively more significant in small diameter catheters, and therefore increases the CMP. The common mode effect can be an order of magnitude higher than the true pressure difference between the measuring locations.

An objective of some embodiments of the invention is to restore the pressure difference between the measuring points from the distorted differential pressure measurement.

In some embodiments of the invention, the distortion, discerned when the distal end of the catheter is in a region with no pressure drop, is used to find a restoration function to correct the distortion. As used herein, the restoration function refers to a transform, for example a linear transform, that acts on a function of time, for example the gauge pressure as a function of time over one or more cardiac cycles, to produce another function of time. Optionally the restoration function is chosen to be a transform that, to good approximation, transforms a measured gauge pressure as a function of time to a function that can be applied to an output signal of a differential pressure sensor (and/or measured by multiple sensors) to find a pressure differential between the sensing locations. Optionally, the restoration function is chosen to be a transform that, to good approximation, transforms the measured gauge pressure as a function of time, to the common mode pressure effect as a function of time, as discerned by the pressure gauge in the absence of any real pressure drop. Optionally, the restoration function is chosen from a set of linear transforms of functions of time, defined by a finite set of parameters, and values of the parameters are found, for which the transformation, applied to the measured gauge pressure as a function of time, provides a good fit to the discerned common mode pressure distortion as a function of time. Optionally, the transform is a linear combination of a finite set of Fourier components of the function of time, and the set of parameters comprises coefficients, optionally complex coefficients, multiplying each of the Fourier components. Alternatively, the transform is a linear combination of different order derivatives of the function of time, possibly time delayed, optionally including the function itself, and the set of parameters comprises coefficients giving a multiplicative factor and a time delay for each of the derivatives. Optionally, finding values of the parameters that provide a good fit comprises finding a least squares fit over the values of the parameters.

Once the restoration function is found, it is optionally used to correct pressure drop measurements when the openings of the two lumens are not protected from the blood flow, but are out of the delivery catheter and exposed to the blood flow. This is done, for example, by measuring the gauge pressure as a function of time, transforming to it with the restoration function to find an expected distortion, and subtracting the expected distortion from the pressure drop as measured by the pressure gauge. The resulting corrected pressure drop is expected to more closely reflect the true pressure drop between the sensing locations. Optionally, this corrected pressure drop is used to find the stiffness of the arterial wall, as described above. Additionally or alternatively, the corrected pressure drop is used to find a reduction in blood flow caused by a stenosis, if the pressure drop is measured across the stenosis. For example, the relative reduction in blood flow caused by a stenosis is found by taking the ratio of excess pressure drop across the stenosis (the increase in pressure drop beyond the pressure drop that would be expected over that distance in the absence of a stenosis) to the total pressure drop across the microvasculature.

Although conventional FFR may also find a relative reduction in blood flow caused by a stenosis, by measuring the pressure drop across the stenosis, in conventional FFR the pressure drop measurements are not sensitive enough to measure the pressure drop across a stenosis unless the stenosis blocks a large fraction of the cross-section of the artery lumen, for example at least 70%, or at least 90%. A potential advantage of using a pressure gauge, and a restoration function to correct for distortion, is that much smaller pressure drop measurements can be made across a stenosis, than with conventional FFR. This is particularly useful for evaluating stenoses that only block between 40% and 70% of the artery lumen cross-section, which could still potentially be dangerous, or stenoses that block less than 40%, which may be less likely to be dangerous in the near term but are even harder to detect.

Another potential advantage of this method over conventional FFR, for evaluating stenoses by measuring the pressure drop across them, is that with conventional FFR it is generally necessary to make the measurements under conditions of hyperemic blood flow, in order to get large enough pressure drops to measure, and in order to have an absolute standard for comparing pressure drops between different subjects. In some embodiments of the method of using a multi-lumen catheter and a pressure gauge outside the body, much smaller pressure drops can be measured. Furthermore, if pressure drops are measured at many different positions along an artery, then the pressure drops at different positions within the same subject can be compared to to each other, and in some embodiments it is not as important to have an absolute standard for comparison. For both these reasons, measurements may sometimes be made without inducing hyperemic blood flow. For example, the pressure drop measurements may be made with a normal level of blood flow, for example less than half of the hyperemic level of blood flow for that artery. This has a potential advantage when making pressure drop measurements in the 10% or 15% of patients in whom hyperemic blood flow cannot be induced. Optionally some embodiments avoid the need to give drugs to induce hyperemic blood flow.

In some embodiments, measurements of pressure drop may be made while moving a probe along a body lumen. As used here the term probe refers to an object that is inserted into something so as to test conditions and/or send back information at a given point. Optionally sensor may be included in the probe. Alternatively or additionally, the probe may send information to a sensor located remotely. For example a probe inserted inside the body may send information to a sensor outside the body. For example, according to some embodiments of the current invention, a differential pressure probe may include a distal end of a multi lumen catheter inserted into a patient. The openings may allow pressure changes on the outside of the opening (inside the patient) to affect the pressure in a lumen. A differential pressure sensor may be located on the proximal end of the lumens optionally remote from the probe and/or optionally outside of the body of the patient.

For example a catheter may be pulled along a blood vessel. Optionally measurements may be made while the probe is moving along the body lumen. For example the probe may move at a rate of between 0.5 and 1 mm/sec and/or between 0.1 and 0.5 mm/sec and/or at a rate slower than 0.1 mm/sec. In some embodiments, measurements for multiple flow conditions may be made while the catheter is stationary and then the catheter may be moved to another portion of the body lumen. Alternatively or additionally, the probe may continue to move while measuring. For example, in a cardiac cycle of 1 Hz, for a probe with measuring locations 5 cm apart moving at a rate of 1.0 mm/sec, the probe may move 0.5 mm between phases of the cycle. The portion of the artery measured at different phases may be 90% the same (90% overlap of the portion of measurements at the two phases). In some embodiments, overlap of 90% or more may be defined as substantially the same portion of the body lumen. In some to embodiments, overlap of 95% or more may be defined as substantially the same portion. In some embodiments, overlap of 99% or more may be defined as substantially the same portion. In some embodiments, overlap of 99.5% or more may be defined as substantially the same portion. Additionally or alternatively, in some embodiments the body lumen may move between measurements. For example coronary arteries may move significantly between phases of the coronary cycle.

In some embodiments, a pressure drop may be measured across a valve. For example pressure drop may be measured for different flow conditions. The results may be used to evaluate the efficiency of the valve in blocking flow when closed and/or in allowing flow when open.

An aspect of some embodiments of the current invention relates to measuring and/or correcting a common mode pressure distortion. For example the distortion may be between two fluid filled lumens of a catheter.

An aspect of some embodiments of the current invention relates to measuring a small pressure differential in-vivo.

An aspect of some embodiments of the current invention relates to measuring a stiffness of a body lumen.

An aspect of some embodiments of the current invention relates to assessing an interventional treatment for example an ablation.

An aspect of some embodiments of the current invention relates to locating a change in a body lumen, for example a stenosis and/or a change in stiffness.

An aspect of some embodiments of the current invention relates to providing a catheter for performing an in-vivo differential pressure measurement.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

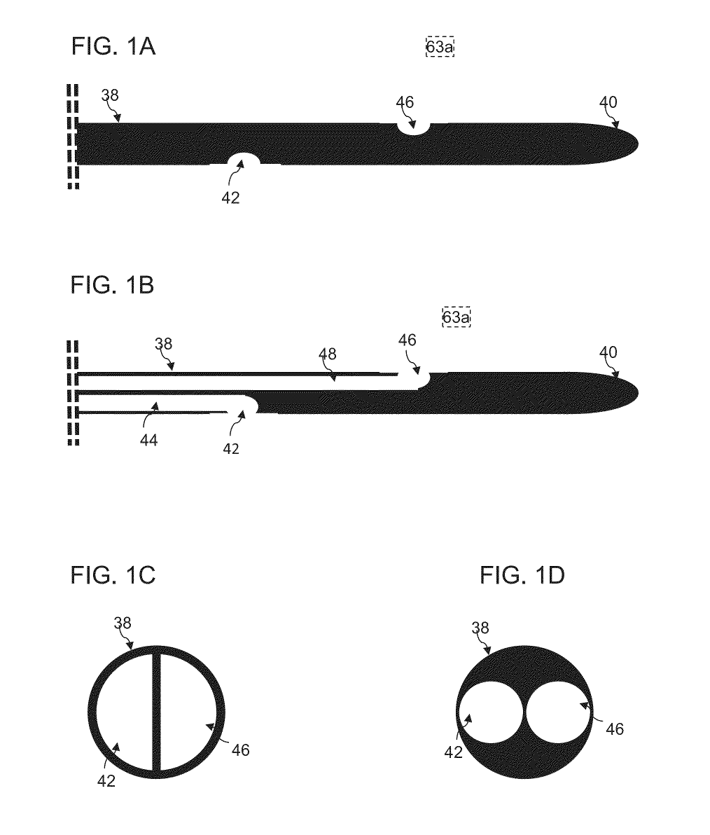

Referring now to the drawings, FIGS. 1A-D schematically illustrates probe 63a (for example a distal end of a catheter 38 comprising two lumens 44 and 48), for making pressure drop measurements in a body lumen, according to an exemplary embodiment of the invention. A side view of probe 63a cut on the symmetry plane is shown in FIG. 1A, to followed by a side cross-sectional view in FIG. 1B, and cross-sectional views perpendicular to the catheter axis, for two different embodiments of the catheter (FIGS. 1C,D). Lumen 44 is in communication with a sensing location, for example a distal opening 42. Lumen 48 is in communication with a sensing location, for example a distal opening 46. The lumens are filled with a fluid, preferably a saline solution, sufficiently incompressible so that they can transmit changes in pressure effectively. The respective distal openings expose the fluid in each lumen to the local blood pressure at the sensing location when the catheter is inserted into an artery. Because opening 46 and opening 42 are at different axial positions along the catheter, and along the artery, the blood pressure at the two openings will generally differ by a small pressure drop, due, for example, to blood flow in the artery along the catheter. Although lumens 44 and 48 need not be straight, making them straight and smooth has the potential advantage that it might reduce the common mode pressure effect.

The diameter of catheter 38 is optionally much smaller than the inner diameter of an artery that it is designed to be used in, so that the catheter can get past a stenosis even if it blocks a large part of the artery lumen, and so that the catheter will not take up a large fraction of the artery lumen, and significantly change the pressure drop just by its presence, and so that the catheter is not likely to be pushed against the wall of the artery, blocking one or both of the distal openings. For example, for use in the left main coronary artery, which has an inner diameter of about 3.5 mm, the catheter is 3 french or less, i.e. less than 1 mm in diameter. If the catheter is pushed against the wall of the artery anyway, blocking or both of the distal openings, as indicated by one or both lumens showing a much smaller peak-to-peak gauge pressure than expected, then optionally the catheter is rotated to pull it away from the wall.

It should be noted that openings 42 and 46 are located at the distal ends of lumens 44 and 48. This means the lumens 44 and 48 end at distal openings 42 and 46, and do not extend further inside catheter 38. This has the potential advantage that air bubbles will not become trapped in a part of the lumen that extends past the opening. Air bubbles can be removed from the rest of the lumen, up to the distal opening, by flushing it, for example with saline solution.

In some embodiments of the invention, other sensors are incorporated into catheter 38, for example a blood flow sensor which can provide information on absolute stiffness of the artery wall as opposed to only relative stiffness. Other sensors, for example intravascular ultrasound sensors, or temperature sensors, may provide additional information on arterial lesions, plaque, and stenoses, which can be used together with data on artery wall stiffness and pressure drop to evaluate such lesions, plaque, and stenoses. Lumens 44 and 48, or another lumen, can be used to inject contrast agents into the artery, for angiography.

In some embodiments probe 63a and/or catheter 38 may be disposable. Alternatively or additionally probe 63a and/or catheter 38 may be reusable.

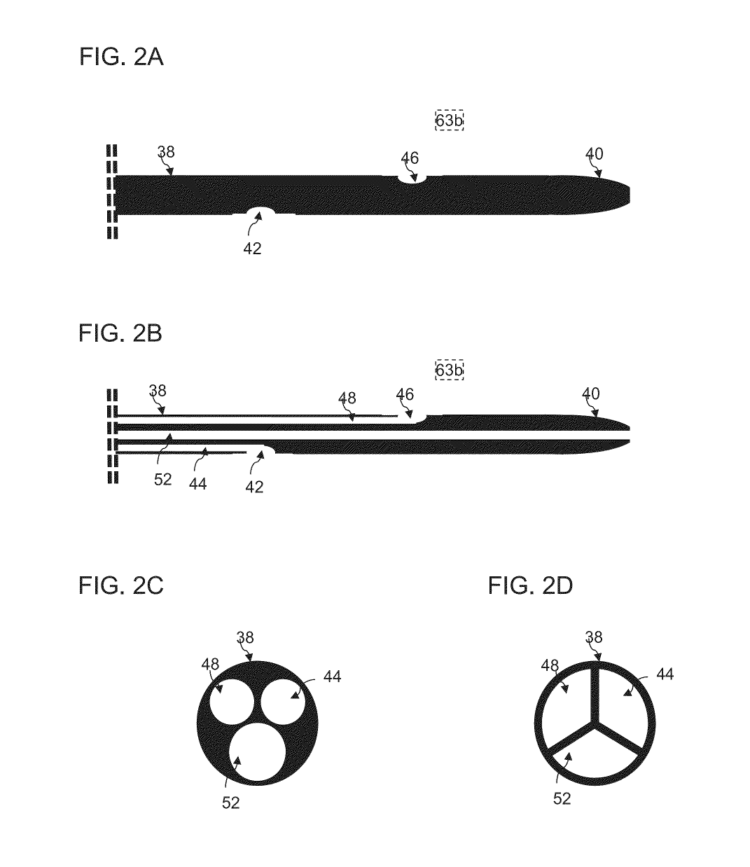

FIGS. 2A-D schematically show a multi-lumen catheter 38, similar to the catheter shown in FIGS. 1A-D, having a third lumen 52, according to an embodiment of the current invention. A side view cut on the symmetry plane of the catheter is shown in FIG. 2A, followed by a side cross-sectional view in FIG. 2B, and cross-sectional views perpendicular to the catheter axis, for two different embodiments of the catheter (FIGS. 2C,D). Third lumen 52 may be used for example for a guide wire. Lumen 52, and any additional lumens that might be present for other purposes, preferably have a port at the proximal end that can be used to flush them, for example with saline solution, to remove air bubbles, at least for safety reasons. Pressure-line lumens (44 and 48) may optionally have, but are not limited to having, the same cross sectional area. Pressure-line lumens (44 and 48) and the guidewire lumen 52 may optionally have the same or different cross sectional areas. In some embodiments, pressure-line lumens (44 and 48) may have larger cross sectional areas than guidewire lumen 52.

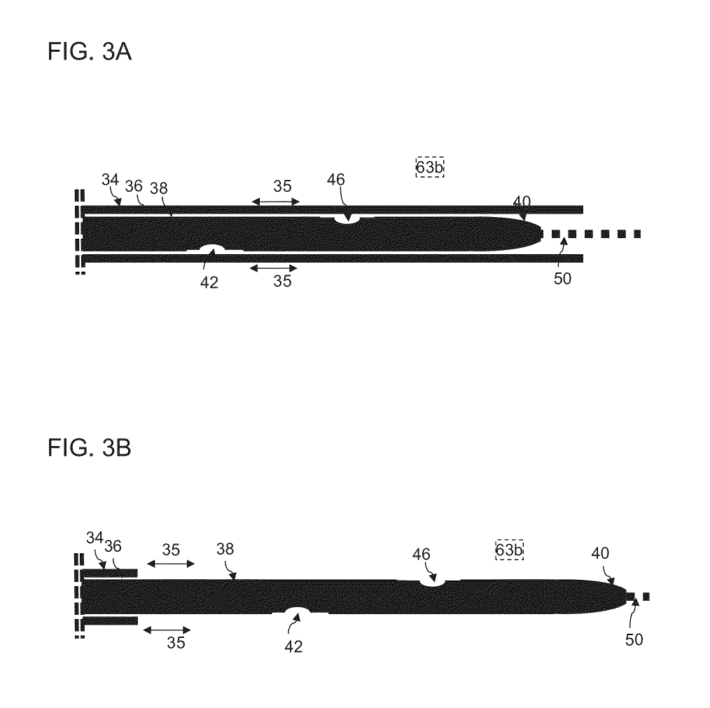

FIGS. 3A,B schematically show relative movement between a multi-lumen catheter, for example catheter 38 shown in FIGS. 2A-D, and a sleeve (for example a delivery catheter 34) according to an embodiment of the current invention. In FIG. 3A, catheter 38 is surrounded by a delivery catheter 34. Optionally multilumen catheter 38 and/or delivery catheter 34 run along a guide wire 50. Catheter 38 is optionally inserted into the artery inside delivery catheter 34, as shown in FIG. 3A. Before measuring the pressure drop, a differential pressure probe 63b, for example the distal end of catheter 38 including sensing locations (opening 42 and 46) may be calibrated in vivo. For example, calibration may take place when delivery catheter 34 is inside a body lumen and the to probe 63b is inside delivery catheter 34. Inside delivery catheter 34, the probe 64c may not be exposed to blood flow in the artery. Without flow, there should be a negligible pressure difference between distal openings 42 and 46. As used herein, the term calibrating a probe may include calibrating parts that are not included in the probe. For example, calibration may include a sensor and or a connecting member associated with the probe. The sensor and/or connecting member may optionally be outside the probe itself. For example a differential pressure probe (for example probe 63b) may consist of a small section of a distal end of a catheter including measuring locations (for example opening 42 and 46 and the catheter between them). Optionally, in vivo calibration of the differential pressure probe includes discerning distortion due to the probe (for example for probe 63b the distal portion of the catheter including the sensing locations that is inserted into the body lumen) and/or a connector (for example lumens 48 and 52) and/or the sensor (optionally located at the proximal end of lumens 48 and 52, outside the body lumen).

In some embodiments, catheter 38 may be pushed forward (to the right in FIGS. 3A,B) out of delivery catheter 34, and/or delivery catheter 34 may withdrawn backward (to the left in FIG. 3A,B) away from catheter 38, exposing catheter 38, for example as shown in FIG. 3B. In particular distal openings 42 and 46 are optionally exposed to the blood flow and pressure drop in the artery. The pressure drop is optionally measured with catheter 38 in the exposed configuration. Optionally, catheter 38 is frequently withdrawn into delivery catheter 34, in order to discern the common mode pressure distortion again. Manipulation of the catheter can sometimes cause changes in the transfer functions of the two lumens, for example due to kinking, and this may change the common mode pressure distortion. In some embodiments, recalibrating the catheter 38, for example after withdrawal into delivery catheter 34, may increase the accuracy of measurements.

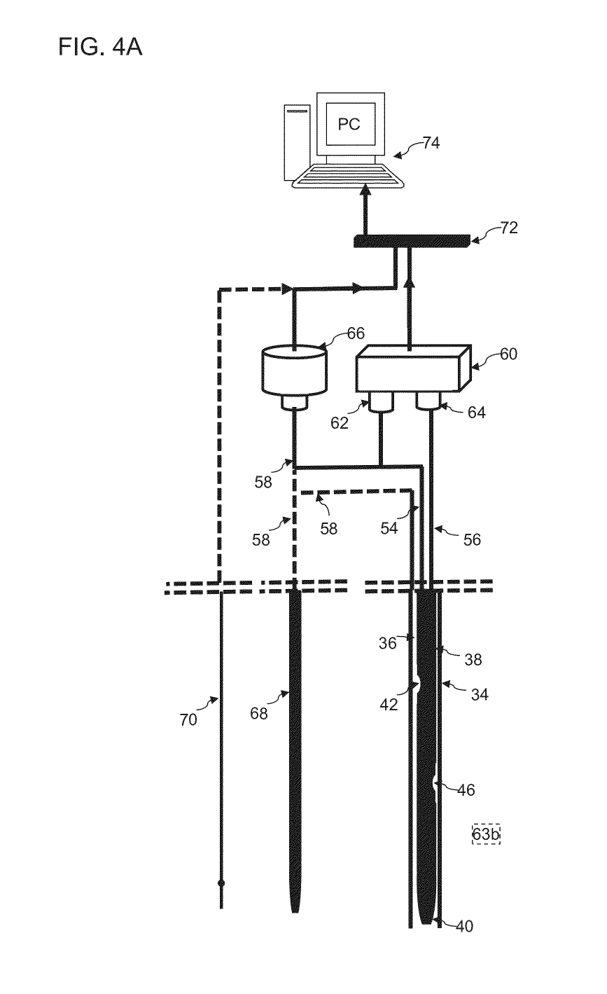

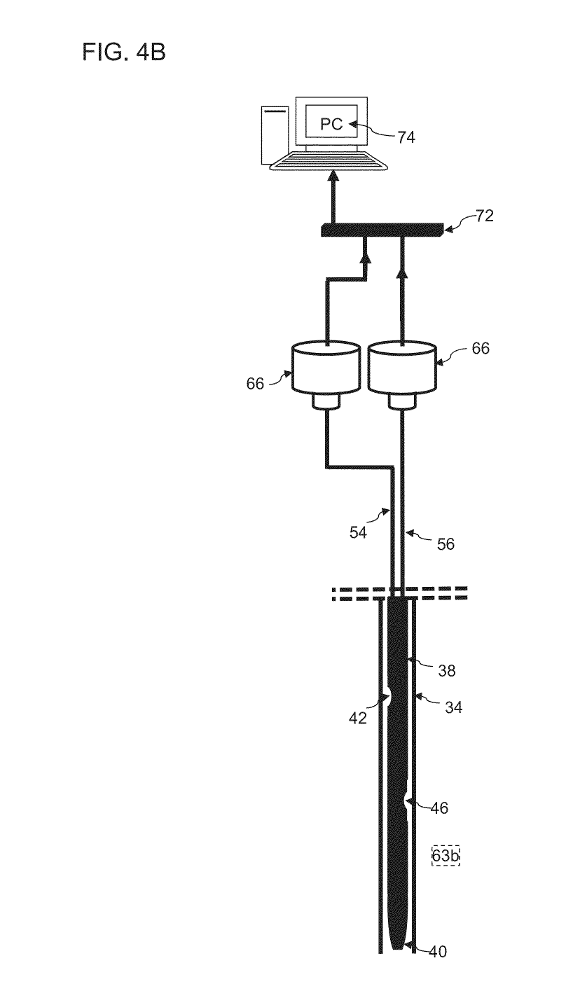

FIG. 4A schematically shows a system for measuring pressure drop in an artery or across a valve, according to an exemplary embodiment of the invention. A multi-lumen catheter 38, similar to multi-lumen catheter 38 in FIGS. 2 and 3, is shown inside delivery catheter 34, inside an artery. Proximal ends of lumens 44 and 48 are connected to extension pressure lines 54 and 56 respectively, also filled with fluid, for example saline solution, and the extension pressure lines in turn are connected to positive to pressure port 62 and negative pressure port 64 of differential pressure gauge 60. Differential pressure gauge 60 is sensitive to small differential pressures, of the precision desired for measuring the pressure drop between distal openings 42 and 46 of the lumens, for example between 0.1 and 0.05 mm Hg, or between 0.05 and 0.01 mm Hg, or between 0.01 and 0.005 mm Hg, or less than 0.005 mm Hg. The range of differential pressure gauge 60 is great enough to cover the expected range of pressure drop between distal openings 42 and 46, as well as the expected common mode pressure distortion. A pressure gauge 66 optionally measures an indicator of the gauge pressure of the artery, and need not have as great a precision as differential pressure gauge 60, but has a range great enough to cover the maximum expected blood pressure, for example up to 250 mm Hg for individuals with high blood pressure. The precision of pressure gauge 66 is, for example, between 5 and 1 mm Hg, or between 1 and 0.5 mm Hg, or better than 0.5 mm Hg. Pressure gauge 66 is connected to one of the extension pressure lines from the multi-lumen catheter and/or to the lumen 36 of delivery catheter 34, and/or to another catheter 68 with a single fluid-filled lumen for measuring pressure located for example in the ostium of the artery where the pressure drop is being measured. Alternatively, an indicator of gauge pressure is measured with a catheter 70 having a pressure gauge directly mounted on it, optionally located in the ostium, instead of or in addition to pressure gauge 66. The pressure data from differential pressure gauge 60, as well as from pressure gauge 66 and/or the pressure gauge on catheter 70, is fed via a data interface 72 into a computer 74, for analyzing the data. Optionally, computer 74 includes software for finding artery stiffness from pressure drop data, and for correcting pressure drop data for common mode pressure distortion, as described below. Optionally, the corrected pressure drop is accurate to within between 0.1 to 0.05 mm Hg, or between 0.05 and 0.01 mm Hg, or between 0.01 and 0.005 mm Hg, a 0.1 mm Hg, or better than 0.005 mm Hg, with a time resolution between 100 and 50 milliseconds, or between 50 and 20 milliseconds, or between 20 and 10 milliseconds, or between 10 and 5 milliseconds, or better than 5 milliseconds. Optionally, in order to measure the pressure drop, it is not necessary to know the true gauge pressure in the body lumen. For example it is not necessary to calibrate the indicator of gauge pressure. A restoration function may optionally be based on a consistent indicator of the pressure in the body lumen that is used in both the calibration stage and in the measuring stage whether or not the actual to gauge pressure is known.

FIG. 4B schematically illustrates a system for measuring pressure drop in an artery and/or across a valve (for example a heart valve and/or venous valve), according to an exemplary embodiment of the invention. The catheter of FIG. 4B optionally uses two gauge pressure transducers 66, one transducer 66 connected to each of the catheter lumens 44 and 48. Gauge pressure may be measured, for example, by simultaneously measuring the gauge pressure in both lumens 44 and 48. The Differential pressure may be obtained by subtracting the reading from the positive pressure line 54 from the reading from the negative pressure line 56. A reference gauge pressure reading for the correction of common mode pressure distortion can be taken in such a configuration from both pressure lines 54 or 56. To get an accurate differential pressure measurement, transducers may need to be highly precise.

In some embodiments probe 63b and/or catheter 38 and/or sensor 60 and/or sensor 66 may be disposable. Alternatively or additionally 63b and/or catheter 38 and/or catheter 68 and/or catheter 70 and/or sensor 60 and/or sensor 66 may be reusable. In some embodiments probe 63b and/or catheter 38 and/or sensor 60 may be supplied as a kit. Optionally the kit also includes catheter 68 and/or catheter 70 and/or sensor 60 and/or sensor 66. Optionally, a kit may include software for use on controller 74.

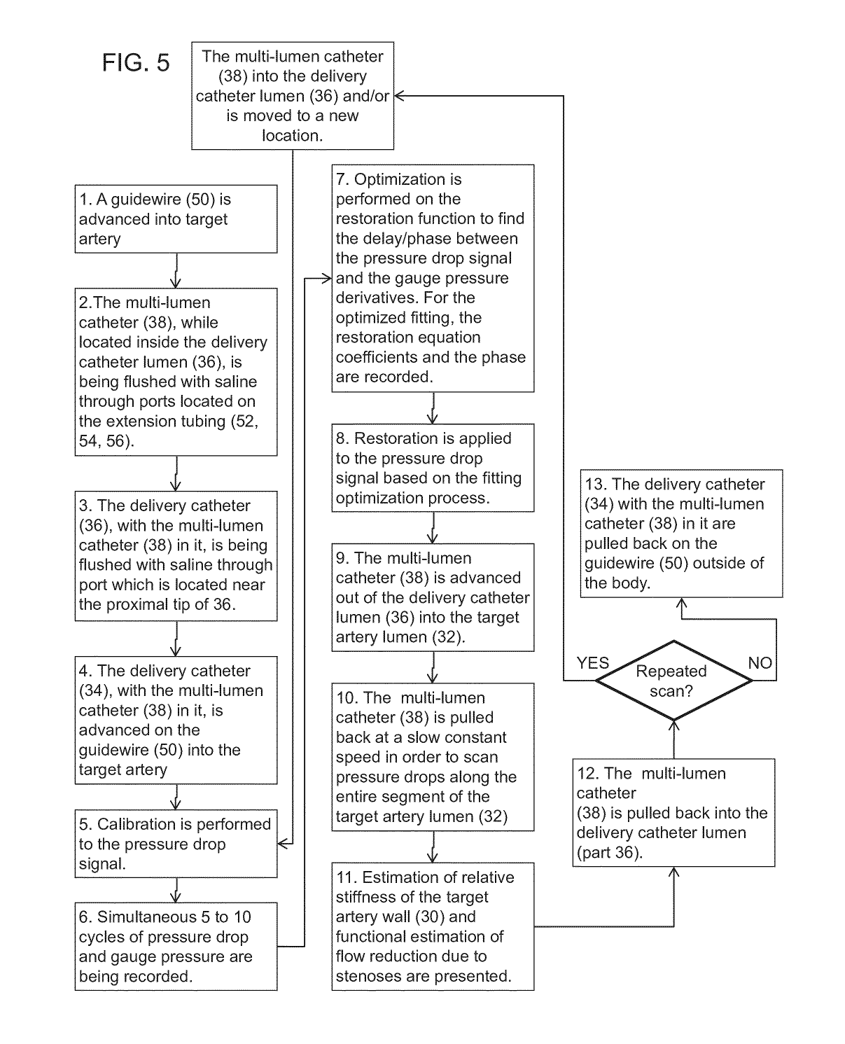

FIG. 5 shows a flow diagram for an exemplary method for measuring relative stiffness of an artery at different portions along the artery, according to an embodiment of the invention. The method of FIG. 5 may, for example, use the system shown in FIG. 4A or 4B. At 1, guidewire 50 is advanced into a target artery. At 2, multi-lumen catheter 38, while located inside a lumen 36 of delivery catheter 34, is optionally flushed, for example with saline solution, through ports locating on one or more of extension lines 52, 54, and 56. At 3, delivery catheter 34 is optionally flushed, for example with saline solution, through a port, not shown in the drawings, located near its proximal end. Flushing the catheters removes air bubbles that may be inside the lumens, which can adversely affect the common mode pressure distortion if they are in lumens 44 and 48. In addition, air bubbles located anywhere in the catheters can be dangerous if they get into the blood stream. Flushing lumens 44 and 48 may also help to remove any floating material blocking distal openings 42 and 46, after catheter 38 is inside the blood stream.

At 4, preferably after flushing the catheters, delivery catheter 34, with multi-lumen catheter 38 inside it, is advanced on guidewire 50 into the target artery. At 5, with catheter 38 still inside delivery catheter 34 where the pressure drop may be negligible, calibration is optionally performed. This is done, for example, by finding a time-averaged pressure drop measured by differential pressure gauge 60, and adjusting differential pressure gauge 60 so that the time-averaged pressure drop goes to zero, or subtracting it from the pressure drop data when the data is analyzed by computer 74. Alternatively or additionally, such a dc offset correction is included in the restoration function, when it is calculated later. At 6, for several cardiac cycles, for example for 5 to 10 cardiac cycles, while catheter 38 is still inside delivery catheter 34 and separated from the blood flow, but is exposed to the blood pressure, data is recorded simultaneously for the pressure drop, from differential pressure gauge 60, and for the gauge pressure, from pressure gauge 66 or from catheter 70.

At 7, a restoration function is found which provides a good fit, optionally a best fit, between data recorded by differential pressure gauge 60, which in the presence of negligible pressure drop may be assumed to be mostly due to the distortion, and the measured gauge pressure transformed by the restoration function. This is done, for example, by assuming that the gauge pressure, transformed by the restoration function, is a linear combination of the gauge pressure and some of its time derivative, for example first and second and optionally some higher order time derivatives, optionally each with a different time delay. A set of coefficients and time delays for each of these terms is then found, which provides a good fit, for example a least squares fit, between the discerned distortion, and the measured gauge pressure transformed by the restoration function.

In some embodiments, the restoration function is based on the idea that the common mode pressure distortion (CMP) may be mainly affected by mismatch in delays between the positive and negative pressure channels. The difference in delay may cause the sensing element inside the differential transducer to sense gauge pressure change first in the channel with the lower delay, and a fraction of time later in the channel with the higher delay. The distortion may be amplified towards positive or negative values, depending on which of the pressure channels has smaller delay. If the difference in delays is large enough (which is very easy to achieve with small diameter catheters), then the CMP can be greater than the pressure drop that is being measured.

The CMP problem is addressed, in some embodiments of the invention, by correlating it with the gauge pressure and/or with the gauge pressure derivatives, as can be seen in Eq.1.

$${P}_{cm} - {m}_{0} + {m}_{1} {P}_{g} + {m}_{2} \frac{d {P}_{g}}{d t} + {m}_{3} \frac{{d}^{2} {P}_{g}}{d {t}^{2}} + … + {m}_{n} \frac{{d}^{n - 1} {P}_{g}}{d {t}^{n - 1}} \qquad Eq . 9$$

Here P_{cm} is the common mode pressure, P_{g} is the gauge pressure, and m₀ to mₙ are the equation coefficients. The measured pressure drop (P_{dm}) is composed from the true pressure drop (P_{d}) and from the CMP (P_{cm}):

P_{dm}=P_{d}+P_{cm} Eq.10

Assuming that both channels of the catheter are connected together in an environment with fluctuating gauge pressure, for example inside the sleeve where there is no blood flow, then theoretically the differential measurement should be negligible (P_{d}=0), therefore:

P_{dm}=P_{cm} Eq.11

In the case of Eq.3 we can find the coefficients m₀ to mₙ to fit P_{cm}, and use it in Eq.2 to estimate P_{d}.

$${P}_{d} = {P}_{dm} - ( \begin{matrix}{m}_{0} + {m}_{1} {P}_{g} + {m}_{2} \frac{d {P}_{g}}{d t} + \\ {m}_{3} \frac{{d}^{2} {P}_{g}}{d {t}^{2}} + … + {m}_{n} \frac{{d}^{n - 1} {P}_{g}}{d {t}^{n - 1}}\end{matrix} ) \qquad Eq . 12$$

The correlation of Eq.1 is optionally increased by adjusting the phase between the measured pressure drop (P_{dm}) and the gauge pressure derivatives

$$( \frac{{d}^{n - 1} {P}_{g}}{d {t}^{n - 1}} ) .$$

The constants m₀ to mₙ for the preferred fitting R-squared value are optionally saved and used for the signal restoration in Eq.4. The weights of each term of Eq.1 are optionally calculated according to Eq.5 and Eq.6.

$$\begin{matrix}{W}_{0} ( t ) = \frac{\vert {m}_{0} \vert}{\vert {m}_{0} \vert + {\sum }_{i = 1}^{n} \vert {m}_{i} \frac{{d}^{i - 1} {P}_{g} ( t )}{d {t}^{i - 1}} \vert} \times 100 & Eq . 13 \\ {W}_{n} ( t ) = \frac{\vert {m}_{n} \frac{{d}^{n - 1} {P}_{g} ( t )}{d {t}^{n - 1}} \vert}{\vert {m}_{0} \vert + {\sum }_{i = 1}^{n} \vert {m}_{i} \frac{{d}^{i - 1} {P}_{g} ( t )}{d {t}^{i - 1}} \vert} \times 100 & Eq . 14\end{matrix}$$

Here wₙ(t) is the percent weight of coefficient mₙ from the total sum of weights of m₀ to mₙ at time t. In an experimental example the highest derivative of P_{g} used in Eq.1 was the second derivative, w₄(t), the percent weight for the term of the third derivative was calculated to be less than 0.2%.

Alternatively, other forms are assumed for the restoration function, for example to the gauge pressure may be transformed by multiplying each of several of its Fourier components by a different amplitude and phase factor. Regardless of the form assumed for the restoration function, a restoration function may be found more accurately if the gauge pressure has strong components at higher harmonics of the cardiac rate, in order to provide information on the transfer function of the lumens over a range of different frequencies.

At 8, the restoration function is used to correct differential pressure gauge data taken again when catheter 38 is still inside a sleeve, for example delivery catheter 34, out of the blood flow, to verify that the common mode pressure distortion is stable, and that the restoration function still works, producing a corrected pressure drop that is close to zero. If the corrected pressure drop is not close to zero, for example at least a factor of 10 lower in amplitude than the uncorrected pressure drop, that may be an indication that the common mode pressure distortion is not stable, but is changing quickly in time, for example due to air bubbles leaking into the lumens or the extension lines, and/or due to kinking of the catheter or the extension lines. Optionally, if this happens, the lumens and extension lines are flushed with saline solution, to try to remove any air bubbles, and/or an attempt is made to remove any kinks, and the procedure is repeated again starting from 5.

At 9, multi-lumen catheter 38 is advanced out of a sleeve, for example, delivery catheter lumen 36, into the target artery, so that it is exposed to the blood flow. Optionally, catheter 36 is advanced as far as it can go in the target artery, for example until the target artery becomes too narrow, or until the target artery branches into smaller arteries that are too narrow. Here, “too narrow” means less than 5 times the diameter of the catheter, and/or less than 3 times the diameter of the catheter, and/or less than 2 times the diameter of the catheter, and/or too difficult to manipulate the catheter in. Advancing the catheter up to the point where the target artery branches into narrower arteries has the advantage that it may be easy to define where that point is, for example by viewing the catheter and the arteries with a fluoroscope or other medical imaging modality.

At 10, the measuring probe (for example probe 63b) is optionally slowly pulled back along the target artery, optionally together with delivery catheter 34, while pressure drop data and indicator of gauge pressure is recorded. The probe may be stopped at location to measure the pressure drop over a single portion of the artery for different phases of the cardiac cycle and/or the catheter may be pulled constantly at a slow rate such that pressure drop is measured at overlapping portions of the artery over different phases of the cardiac cycle. For example, the movement of the catheter over a single cardiac cycle may be less than 1% the length of the measurement interval so that measurement at different phases is over substantially the same portion of the artery (with an overlap of over 99%). Alternatively or additionally, the substantially same portions of the artery may have an overlap of over 95%. Alternatively, a differential may be computed of the pressure drops from overlapping portions of the artery to estimate a local stiffness and/or flow resistance. The pressure drop data may optionally be corrected using the restoration function, in real time as the data is recorded, and/or later. Pressure drop corrections may optionally be calculated by computer 74. Pulling the catheter slowly back along the artery, while taking this data, allows pressure drop data to be recorded at several different portions of the artery. Pressure drop data from different portions may be compared to find a relative stiffness of the artery wall at different locations. Relative comparison can be made even if the blood flow is not measured. Optionally, different portions of the artery wherein pressure drop is measured may all have the same length (for example when the distance between sensing locations of the probe remains fixed). Optionally, the blood flow in each phase of the coronary cycle is assumed to be constant during the time the catheter is being pulled back. Optionally, the blood flow in each phase of the cycle may be assumed to be constant when the gauge pressure in each phase of the cycle remains about the same as a function of cardiac phase, and/or when there are no significant branches coming off this portion of the artery. Alternatively, the pressure drop and gauge pressure are measured in only one portion of the artery, without drawing back the catheter, which has the potential to advantage that the common mode pressure distortion may be less likely to change if the catheter is not moved. In some embodiments an absolute stiffness of the artery wall can be found for one or more portions of the artery. For example, absolute stiffness may be computed based on the pressure drop for two different flow conditions and the blood flow rate. Optionally blood flow is measured, for example, using an intravascular flow sensor attached to the catheter, for example a thermal flow sensor, or using a Doppler ultrasound sensor, or a Doppler laser sensor, or MRI imaging which can measure blood flow velocity.

At 11, an estimate is made of the relative stiffness of the target artery wall, as a function of position along the artery, by analyzing the pressure drop data and/or the gauge pressure data. The pressure drop is also optionally used to estimate a reduction in blood flow due to any stenoses that are encountered as the catheter is pulled back along the artery. Alternatively, these estimates are made later, after the catheter is removed from the body, using the pressure drop data and gauge pressure data that was recorded, but a potential advantage of making the estimates in real time, while the catheter is still in the artery, is that data can more easily be taken again if it is seen that the data was not good.

To estimate the relative stiffness of the artery by analyzing the pressure drop, the Poiseuille equation for steady laminar flow in a rigid tube is optionally used. This equation shows an inverse relation between the pressure drop and the diameter of the tube:

$$Δ P = \frac{128 μ L Q}{π {D}^{4}} \qquad Eq . 15$$

Here Q is the flow rate, D is the diameter of the tube, μ is the dynamic viscosity, ΔP is the pressure drop, and L is the tube length over which the pressure drop is measured. Change in the diameter D inversely and by a power of 4 affects the pressure drop ΔP. Therefore, for a given pulsatile flow rate, different distensibility values of the artery lumen will yield different maximal pressure drops; lower distensibility of the artery during the maximal peak flow will yield higher pressure drop since the lumen cross sectional area is smaller than normal. Such difference between two arterial segments is expected to be seen mainly in the high value regions of the flow cycle, e.g. systolic phase, since then it is more likely that we will get significant difference in diameters. The Poiseuille model may be appropriate for steady flow in a rigid tube. Optionally, the Poiseuille model may provide an acceptable estimation of the pressure drop changes as a function of the tube distensibility in a flexible tube with changing flow rate. For example, the Poiseuille model may be used when the diameter deformations are small and/or there is low frequency of pulsatility. Alternatively, for finding the distensibility of the artery wall, the Womersley equation may be used. The Womersley equation may be used for pulsatile flow in some cases.

TABLE 1
Estimation of pressure drop and peak to peak values as a function
of the arterial distensibility in LMCA. The calculation is based
on the poiseuille equation (Eq. 9) nominal diameter D = 3.5
mm (during minimum flow), dynamic viscosity μ = 0.004
Pa · s, minimum flow is 0 ml/min, maximal flow is 110 ml/min,
and the length of the arterial segment is L = 1 cm. 102% distensibility,
for example, means 2% distensibility during maximal peak of the
flow cycle. % change from rigid means the percent change of the
pressure drop ΔP for any % distensibility at maximal peak flow
compared to 0% distensibility at maximal peak flow. % change from
previous means the percent change of the pressure drop ΔP during
maximal peak flow between two consecutive distensibility values;
e.g. the expected change in pressure drop between 10% distensibility
and 9% distensibility, for the flow conditions mentioned above, is 3.59%.

| | | | | % change | % change | Peak |
|---|---|---|---|---|---|---|
| | Distensi- | | ΔP | (from | from | to Peak |
| Flow | bility [%] | | [mmHg] | rigid) | previous) | [mmHg] |
| Peakₘᵢₙ | | 100 | 0.000 | | | |
| Peakₘₐₓ | Rigid | 100 | 0.149 | | | 0.149 |
| | Flexible | 101 | 0.144 | 4.06% | 3.90% | 0.144 |
| | | 102 | 0.138 | 8.24% | 3.86% | 0.138 |
| | | 103 | 0.133 | 12.55% | 3.83% | 0.133 |
| | | 104 | 0.128 | 16.99% | 3.79% | 0.128 |
| | | 105 | 0.123 | 21.55% | 3.76% | 0.123 |
| | | 106 | 0.118 | 26.25% | 3.72% | 0.118 |
| | | 107 | 0.114 | 31.08% | 3.69% | 0.114 |
| | | 108 | 0.110 | 36.05% | 3.65% | 0.110 |
| | | 109 | 0.106 | 41.16% | 3.62% | 0.106 |
| | | 110 | 0.102 | 46.41% | 3.59% | 0.102 |

Table 1 depicts an exemplary estimation of expected pressure drop values as a function of different distensibility values (0-10%) under conditions of physiological flow in a left main coronary artery, based on the Poiseuille equation. The difference in maximal pressure drop due to 1% change in arterial distensibility is approximately 3.7%, and 0.005 mmHg in absolute values. Such differences are very small and may be beyond the accuracy, or resolution, of commercially available catheter tipped pressure transducers. Nevertheless, there are commercially available wet/wet differential pressure transducers for very low pressure ranges, which are capable of measuring these changes. Such low pressure differential transducers, even if they cannot be deployed on a catheter tip, can be connected to a fluid filled catheter for acquiring the measurements.

At 12, catheter 38 is pulled back, as indicated schematically with arrow 35, into lumen 36 of delivery catheter 34. If more data is to be taken in that artery, then delivery catheter 34 is optionally moved to a new location if desired, and the procedure is repeated, optionally starting from 5, zeroing the pressure drop signal, as described above. Optionally, measuring the common mode pressure distortion, by withdrawing catheter 38 back into delivery catheter 34, and finding the restoration function, are repeated whenever the catheter is moved very far, for example more than 5 cm, or more than 10 cm, and/or every few minutes for example once in a time interval ranging between 1 to 3 minutes and/or ranging between 3 to 5 minutes and/or ranging between 5 to 10 minutes and/or ranging between 10 minutes and 20 minutes, in case the transfer functions for the probe might have changed since the last time the common mode pressure distortion was measured.

At 13, when all the data has been taken, delivery catheter 34, with catheter 38 inside it, is pulled back on guidewire 50 and removed from the body.

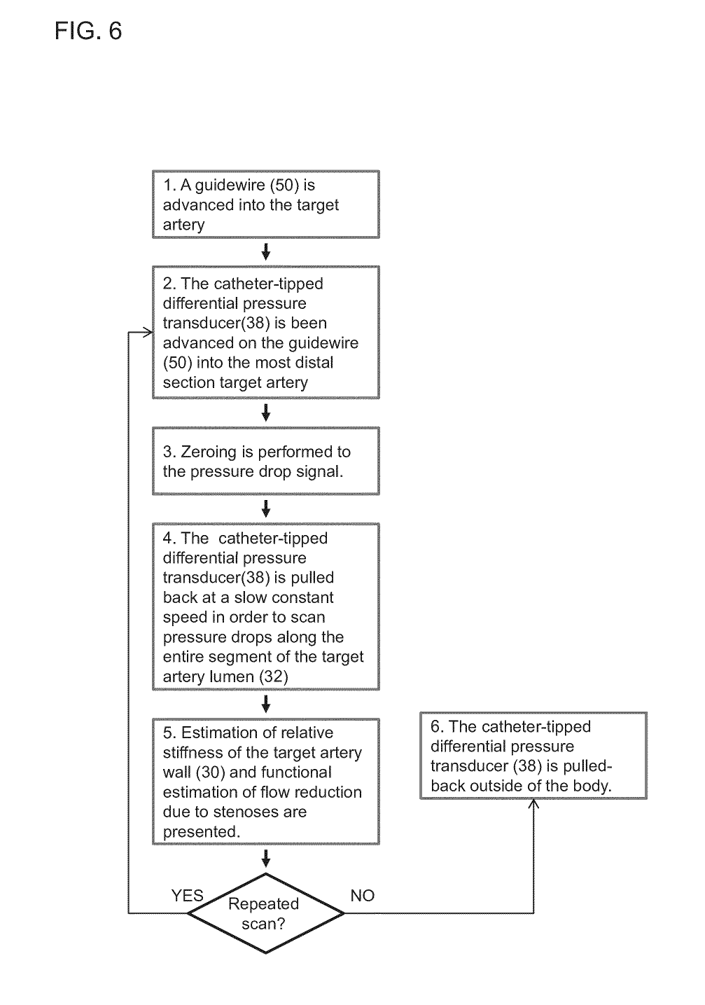

FIG. 6 shows a flow chart for an alternative method of measuring relative stiffness of an artery wall at different locations along its length, using a catheter with a differential pressure sensor mounted directly on it, to measure a pressure drop along the artery. If such a differential pressure gauge, or two separate pressure gauges, can be made with sufficient sensitivity and precision to measure pressure drops accurately over to relative small distances along an artery, for example less than 5 cm, or less than 3 cm, or less than 1 cm, then pressure drops can be measured without any need to use a restoration function to correct them for common pressure mode distortion, and these pressure drops can be used to find a relative stiffness at different locations along the artery. Optionally, calibration for other distortion may be performed using the method of the example of FIG. 6.

Another possible application for the invention, is measurement of pressure drop over a valve (e.g. one of the heart valves, venous valve etc.). In some embodiments, pressure drop measured over a valve may be used is to assess the valves functionality while open (if the pressure drop is considered significant, it means that the valve apply too much resistance for flow through it) and/or the integrity of the valve when closed. Pressure drop over aortic valve (for example) are quite large (can reach the scale of 80 mmHg). The SNR of measurements over the aortic valve (signal to noise ratio) may in some embodiments be larger than the SNR when measuring coronary, renal, and carotid arteries. SNR may be lower, for example for valves over which the drop is lower (e.g. other heart valves (e.g. pulmonary), venous valves, etc.). In some embodiments, the pressure drop over a valve might need to be measured over a distance larger between 5-10 cm. Alternatively or additionally, the pressure drop may be measured of a distance between 1-5 cm.

At 1 in FIG. 6, a guidewire is advanced into a target artery. At 2, a catheter, tipped with a differential pressure sensor, is advanced into an artery, optionally to the most distal point at which pressure drop measurements will be made. The differential pressure sensor measures a difference in pressure between two points along the catheter, and hence along the artery, separated for example by less than 5 cm or less than 3 cm or less than 1 cm. Having the sensors closer together makes it possible to measure differences in artery wall thickness with better spatial resolution along the artery, but having the sensors further apart increases the pressure difference and makes it easier to measure.

Optionally, the catheter also has a gauge pressure sensor, possibly with less sensitivity but with a greater range than the differential pressure gauge. Alternatively, there is a gauge pressure sensor mounted on a separate catheter, for example located in the ostium of the target artery. Alternatively, instead of a differential pressure sensor and to a separate gauge pressure sensor, there are two gauge pressure sensors, mounted at different positions along the catheter, and the pressure drop is measured by the difference in gauge pressure measured by the two gauge pressure sensors. However, using a single differential pressure sensor has the potential advantage that it is likely to have much greater sensitivity, if the gauge pressure sensors are capable of measuring pressures as high as typical blood pressures, which are much higher than typical pressure drops in an artery.

At 3, the pressure drop data is optionally zeroed. This is done, for example, by measuring the pressure drop when the catheter is inside a sleeve (for example a delivery catheter), separated from the blood flow. At 4, with the differential pressure sensors exposed to the blood flow, pressure drop data is taken, and gauge pressure data is taken, optionally while pulling the catheter slowly back along the artery, in order to measure the pressure drop at several different locations along the artery. At 5, the pressure drop data and gauge pressure data, as functions of phase in the cardiac cycle, are used to estimate stiffness of the artery wall at different locations along the artery, and optionally also to estimate the reduction in blood flow due to any stenosis that is present in the artery. At 6, if no further data is to be taken, the catheter is removed from the body.

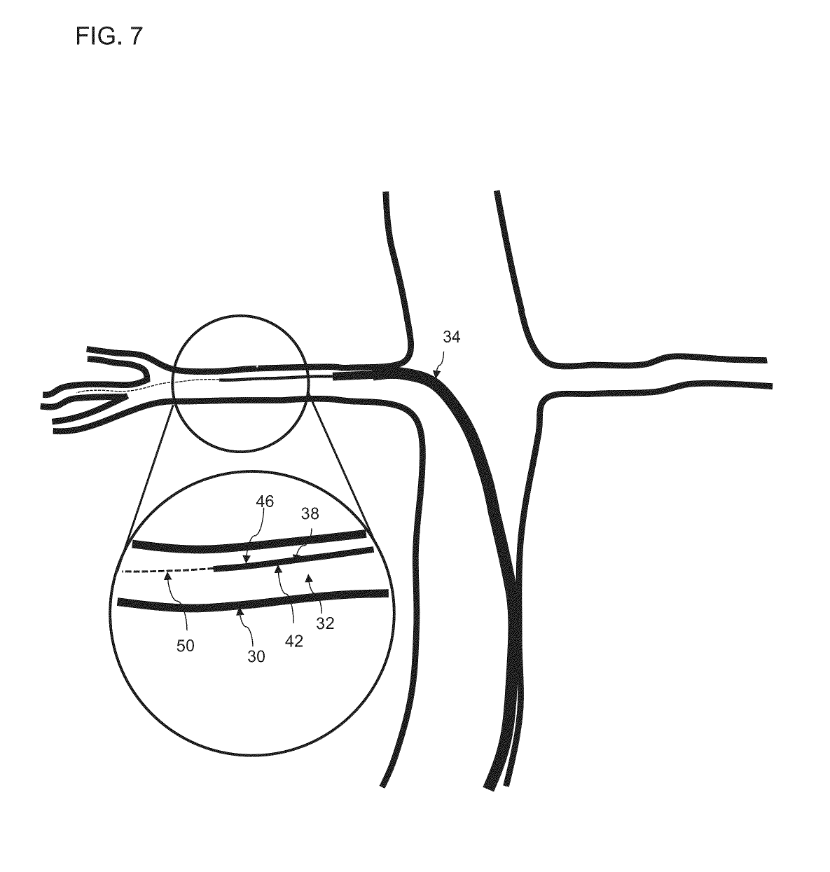

FIG. 7 schematically illustrates an example in the renal artery, during a procedure for treating renal denervation, according to some embodiments of the current invention. Optionally, system may be used to assess an intervention and/or treatment. For example the system may be used to verify that a procedure, for destroying a renal nerve with thermal ablation, was successful. Optionally, the measuring can be done using the method shown in FIG. 5 and/or FIG. 6. In some embodiments, delivery catheter 34, with multi-lumen catheter 38 inside it, is inserted into lumen 32 of renal artery 30, via the aorta, running along guidewire 50. A pressure drop is measured using distal openings 42 and 46, which connect via lumens in catheter 38 to a differential pressure gauge located outside the body, as for example in FIG. 4A or 4B. Optionally the system may include an interventional device to perform the intervention. For example, in renal denervation the interventional device may include an ablation probe including for example electrodes on catheter 38 and/or another catheter that may optionally be introduced into the body lumen via delivery catheter 34. Thermal ablation may be used to destroy a renal nerve. In some embodiments, as a side effect from the to heat, the renal artery wall becomes stiffer and/or other changes in flow. Optionally, measuring changes in flow and/or the stiffness of the renal artery may verify that the thermal ablation of the nerve was performed. Stiffness measurements may be made, for example, as described above in FIG. 5. Measurements of changes in flow may include differential pressure measurements made with for example a differential pressure catheter as described in embodiments herein above or below. Optionally quality of ablation may be assessed based on the absolute stiffness of the artery wall for example by comparing relative stiffness before and after ablation. Alternatively or additionally, the relative stiffness is found after the treatment, at different locations along the renal artery. From the change in relative stiffness it may be possible to see whether, at the location where the heat treatment was applied, the relative stiffness is greater than areas that were not ablated, and/or how much greater it is. The increase in stiffness of the renal artery can then be compared to the increase in stiffness that would be expected from the thermal ablation of the nerve, to verify that the thermal ablation was done with the proper amount of energy deposited in the tissue. Alternatively or additionally, the stiffness may be compared at the same location. For example, the quality of ablation may be assessed based on changes over time of the relative stiffness. For example, the ablation may be assessed based on whether the relative stiffness increased, and/or by how much, after the thermal ablation of the nerve. Optionally, relative stiffness is assessed assuming that the blood flow did not change after the thermal ablation procedure. In some embodiments of the invention, the stiffness of the renal artery wall is found repeatedly from pressure drop measurements during the thermal ablation procedure, and the thermal ablation procedure is stopped once the renal artery wall stiffness has increased by a predetermined amount. This method can help to verify that the thermal ablation has been adequate to destroy the nerve, but has not continued long enough to cause any collateral damage to the renal artery and other nearby tissues.

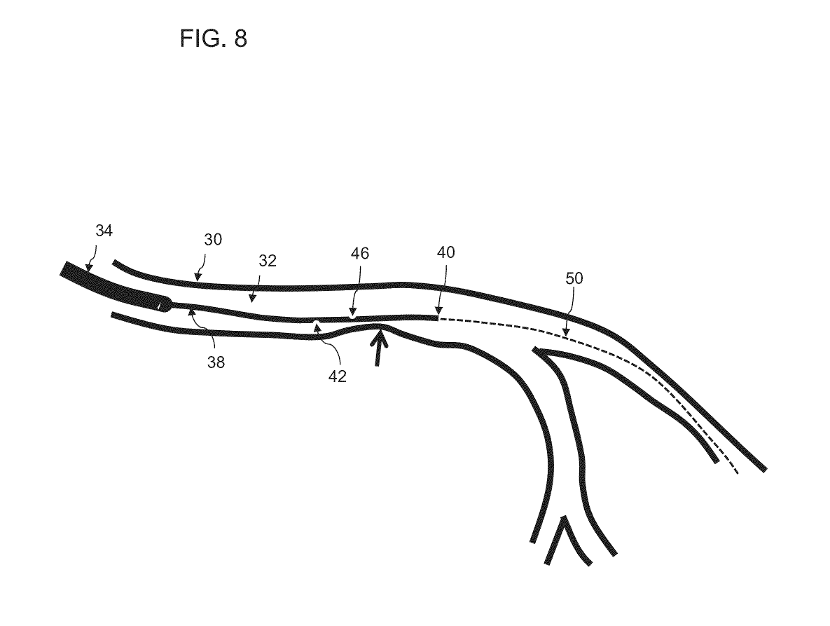

FIG. 8 schematically illustrates system for measuring the pressure drop in different portions along a body lumen, and estimating the relative wall stiffness at locations along the body lumen according to some embodiment of the current invention. Optionally, the system of FIG. 8 may be used according to the method as described in FIG. 5, applied for example to a left main coronary artery 30. For example, the system of FIG. 8 may be used to find and/or to evaluate a stenosis. For example, a higher stiffness to at the location of the stenosis may indicate an atheroma at that location, while a lower stiffness there may indicate vulnerable plaque. The evaluation may optionally help in deciding whether to treat the stenosis, for example with a stent. In some embodiments, the stenosis may be detected by its higher than normal pressure drop, even if it is not visible in an angiogram. In some embodiments, a reduction in blood flow may be estimated. The measured change in blood flow may be used to estimate how much the stenosis is reducing the blood flow in this artery. This information may also be useful in deciding whether to treat the stenosis.

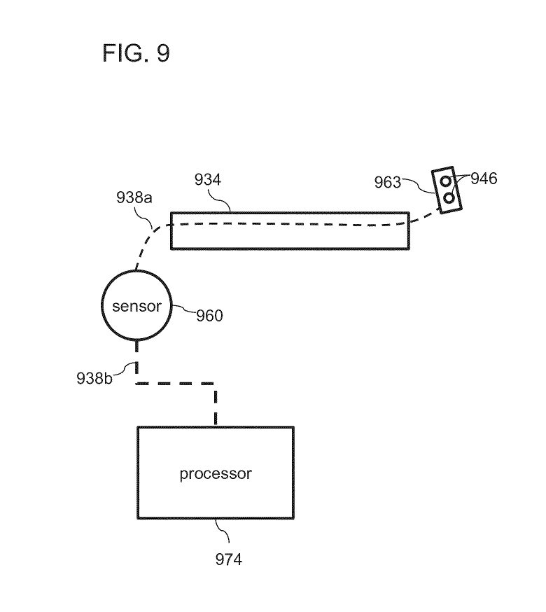

FIG. 9 is a block illustration of a system for measuring a pressure drop in a body lumen according to an embodiment of the current invention. The system may optionally include a differential pressure probe 963. For example, differential pressure probe 963 includes two measurement locations 946. Probe 963 is optionally inserted into the body of a subject. Measurement locations 946 optionally sample the pressure at two locations inside the body lumen. The system optionally includes a communication channel 938a facilitating communication between probe 963 and a pressure sensor 960. Alternatively or additionally sensor 960 may be included in probe 963. Sensor 960 optionally communicates over a communication channel 938b with a processor 974. Optionally the system may include a delivery device 934.

In some embodiments probe 963 may include the distal end of a multilumen catheter (for example catheters 38 of FIGS. 1A-2D). Sensing locations may include openings into lumens of the catheter (for example openings 42 and/or 46). Communication channel 938b may include the lumens of the catheter. Optionally sensor 960 is located near the proximal end of the catheter and/or measures a differential pressure between the lumens of the catheter. Delivery device 934 may include a sleeve that may optionally shield probe 946 from flow during calibration. Delivery device 934 may include a delivery catheter.

In some embodiments, probe 963 include sensor 960. Communication channel 983b may include wire on a delivery catheter and/or a wireless channel (for example probe 963 may include a capsule probe. Delivery device 934 may include for example a delivery catheter and/or a sleeve. In some embodiments, probe 963 may be disposable. Alternatively or additionally, probe 963 may be reusable. In some embodiments, sensor 960 may be disposable. Alternatively or additionally, sensor 960 to may be reusable. In some embodiments, probe communication channel 938a may be disposable (for example including a multilumen catheter and/or a delivery catheter). Alternatively or additionally, probe 963 may be reusable. In some embodiments, delivery device 934 may be disposable. Alternatively or additionally, delivery device 934 may be reusable.

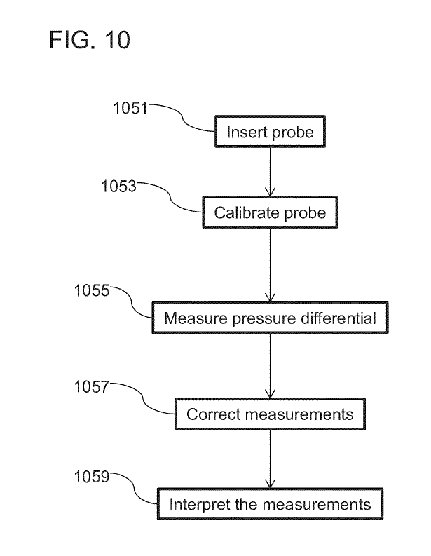

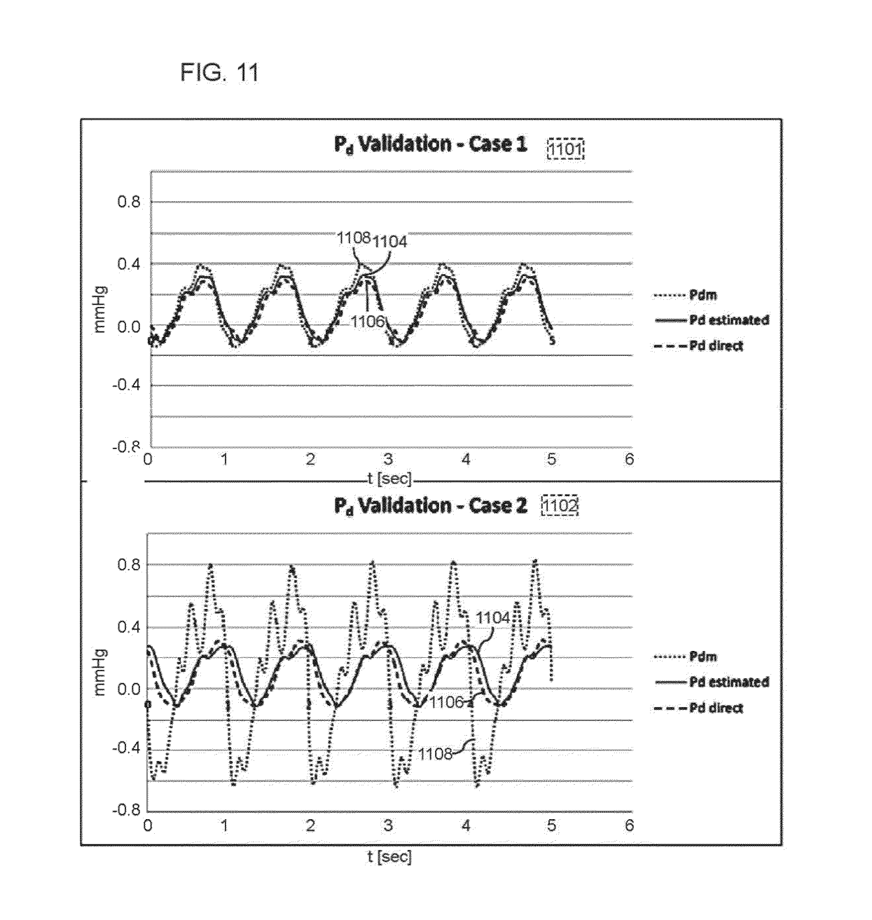

FIG. 10 is a flow chart illustration of a method of measuring a pressure differential according to some embodiments of the current invention. A probe may be inserted 1051 into a body lumen of the patient. For example, the probe may be inserted using a delivery catheter. Alternatively, the probe may be self mobile or flow with a body fluid. In some embodiments the probe may be calibrated 1053. Optionally the probe is calibrated in vivo. Calibration optionally includes calibrating elements of the probe and/or a communication channel and/or a sensor. In some embodiments, the probe may include a sleeve. The sleeve may protect measurement locations on the probe from flow in the body lumen during calibration. The sleeve is optionally retracted during measurement. For example, the sleeve may include a delivery catheter. Alternatively or additionally, the sleeve may be a part of the probe that may be retracted, for example by an actuator and/or the sleeve may be self retracting (for example the sleeve may dissolve over time). Alternatively or additionally, calibration may be performed in-vivo without a sleeve. Alternatively or additionally, calibration may be done outside of the patient, for example before and/or after inserting 1051 the probe.

In some embodiments, a pressure differential may be measured 1055 by the probe. The pressure measurements are optionally corrected 1057. For example, correction may be based on the results of calibration. The measurements may be interpreted 1059. For example, the measurements may be used to compute the stiffness of the body lumen and/or FFR and/or to assess a treatment (for example an ablation of the lumen) and/or condition of the lumen (for example a stenosis).

It is expected that during the life of a patent maturing from this application many relevant blood pressure sensors and gauges, and blood flow sensors, will be developed and the scope of the terms pressure gauge, pressure sensor, and flow sensor is intended to include all such new technologies a priori.

As used herein the term “about” refers to ±10%.

The terms “comprises”, “comprising”, “includes”, “including”, “having” and to their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, to described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Test of Finding Arterial Wall Stiffness from Pressure Drop Measurements

Blood flow, pressure drop and arterial distensibility may be indicative of the coronary arteries' health and atherosclerosis severity. In some embodiments pressure drop is measure to assess of a stenosis severity. Optionally, arterial distensibility is measured to indicative of the arterial stiffness and/or to provide information on the arterial wall composition.

In a first example, the correlation between the fluid pressure drop and the arterial distensibility was investigated for different cases of stenosis severity and flow. The investigation methods included: (1) in-vitro wet experiment on silicone mock arteries, (2) Numerical Fluid Structure Interaction (FSI) simulations, and (3) ex-vivo experiments on arteries of an animal model. Preliminary in-vitro experiments included a non-stenotic arterial model with three different distensibility cases: 10% (high), 4% (intermediate), and impaired (1%). A uniform 0.2 Hz sinusoidal flow rate was applied to mimic coronary pressure drops. Statistical analysis was performed offline on the pressure drop products. Significant changes were observed in the preliminary results in the peak-to-peak pressure drop (dP-P2P) between the different distensibility cases, with 0.926±0.016 mmHg for the 10% distensibility case, 1.156±0.011 mmHg for the 4% distensibility to case, and 2.59±0.029 mmHg for the 1% distensibility case (p<10-5 between all cases). The significance of the differences in peak-to-peak pressure drop between the different distensibility cases is high. The preliminary results may have implications for the relation between the pressure drop and the radial distensibility, and on the potential to produce both functional and bio-mechanical data from pressure drop measurement only.

### Test of Correcting Pressure Drop Measurement Using Restoration Function

Pressure drop was measured in a 5 mm rigid straight tube model over a distance of 3 cm using a differential pressure transducer. The flow rate and gauge pressure in the flow loop were set to 120/80 mmHg and 0-300 ml/min respectively, using 1 Hz pulsations. The working fluid was 37° C. water. The pressure drop measurements were taken once using a 5 french fluid-filled catheter, which catheterized the model, and then via direct connection of the transducer to two ports in the model, as a reference for validation. The distorted pressure drop signal 1108, taken from the fluid-filled catheter, was processed and recorded online using our restoration function, and was then compared to the reference signal 1106 which was directly recorded from the model. Two validation cases were tested: Case 1 (1101) mild, and Case 2 (1102) severe CMP distortions. Case 1 (1101) was achieved using several techniques for air bubble removal from the working fluid. Case 2 (1102) was achieved by introducing a micro-bubble of air into one of the differential pressure transducer ports.

FIG. 110 depicts the validation of our pressure drop restoration method. It can be seen that the restored signal 1104 (P_{d} estimated) is in a very good agreement with the reference signal 1106 (P_{d} direct) both in Case 1 (1101) where the CMP distortion was low (0.17 mmHg peak to peak), and in Case 2 (1102) where the CMP distortion was severe (1.49 mmHg peak to peak).

The results suggest the usefulness of our restoration function, as it successfully estimated online the real pressure drop signals 1104 from highly distorted signals 1108 measured with a small fluid-filled catheter. Case 2 (1102) of severe distortion is may be more realistic for catheterization measurements in-vivo, as air bubbles removal in-vivo may sometimes be less efficient than in-vitro. Using our restoration function, it appears from these results that pressure drop measurements can be reliably and accurately taken using fluid-filled catheter system.

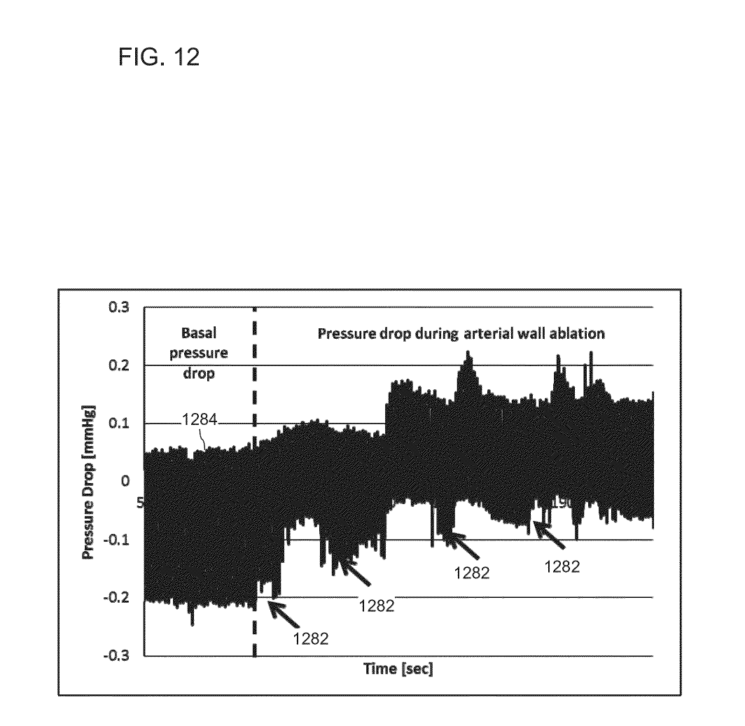

FIG. 12 illustrates results of measuring the effects of ablation on pressure drop in an artery according to some embodiments of the current invention. The example measurements 1284 of FIG. 12 were made ex vivo on a porcine carotid artery. FIG. 12 illustrates measured pressure drops over time. Arrows 1282 illustrate the times at which thermal ablation occurred (a heated steel instrument to the outside of the artery for 10 seconds). Flow parameters included a systemic pressure of 121/72 mmHg at a rate of 60 cycles per minute at an average flow rate of 100 ml/min. In vivo ablation energy may be applied by an ablation device including for example an electrode (for example for radio frequency and/or microwave frequency ablation) and/or a high intensity ultrasound device and/or a laser and/or other techniques. The measured pressure drop show significant sensitivity to ablation. In some embodiments, changes in arterial compliance are detected by changes in pressure drop. In some embodiments spasm (shrinkage) of an artery, for example as a response to the thermal ablation, may be detected by measuring the changes in pressure drop that they cause.

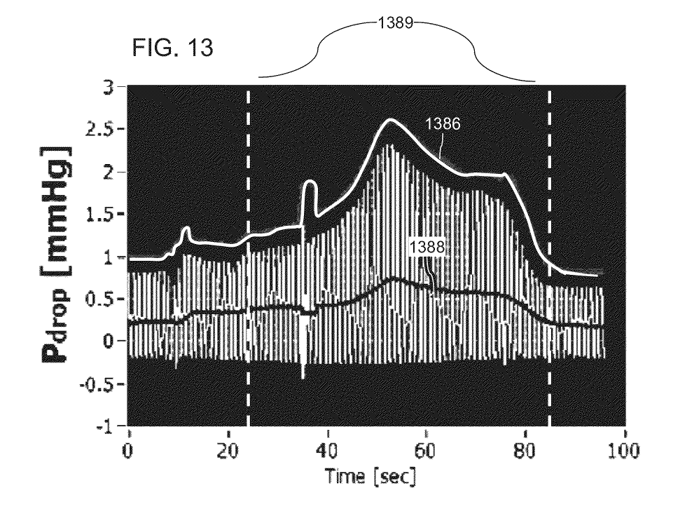

FIG. 13 illustrates results of measuring pressure drop for two flow conditions along a lumen with variable distensibility according to some embodiments of the current invention. In the in-vitro example a differential pressure probe on a catheter (the experimental device was similar to the embodiment of FIG. 1) was pulled back along a silicone mock artery with a mildly impaired distensibility regions having a Functional Flow Reserve indicator (FFR) of greater than 0.99 (FFR>0.99). The impaired distensibility regions are illustrated between dashed lines 1389. The diameter of the mock artery was 4.8 mm During the experiment the systemic pressure was 130/70 mmHg, and the average flow rate was 152 ml/min Local pressure drops are markedly affected by the arterial stiffness, and these changes in pressure drop can be detected using methods and devices according to embodiments of the present invention even in small to intermediate size stenoses, where the FFR values remains greater than 0.99 (FFR>0.99). In the example of FIG. 13 ‘Drop’ indicates the pressures drop; for example peak-to-peak 1386 and mean 1388 values and/or their difference.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for measuring stiffness of a body lumen comprising:
a probe including two measuring locations;
a pressure gauge, that generates a signal indicative of the differential pressure between said measurement locations; and
a controller, adapted to compute a pressure drop between said measuring locations based on the signal of the pressure gauge, for each of at least two different flow conditions, and to find a relative stiffness of the body lumen from the pressure drops.

2. The system of claim 1, further comprising:
a pressure-measuring catheter including:
a fluid-filled first lumen and wherein a first of said measurement locations includes an opening from said first lumen to an outside surface of the pressure-measuring catheter and the pressure measuring catheter also includes
a fluid-filled second lumen, and wherein a second of said measurement locations includes an opening from said second lumen to the outside surface of the pressure-measuring catheter, both said openings being inside the body lumen when the pressure-measuring catheter is inserted, the opening of the first lumen being more distal than the opening of the second lumen, and both said first lumen and said second lumen having proximal ends outside the body when the pressure-measuring catheter is inserted;
and wherein said pressure gauge measures a pressure differential at one or more of the proximal ends of the first and second lumens and said signal depends on said measured pressure differential.

3. The system of claim 1, wherein said body lumen is a blood vessel and the pressure-measuring catheter is configured for insertion into the blood vessel, and wherein said controller is adapted to find a pressure drop from the differential pressure signal for said at least two different flow conditions including at least two different phases of a cardiac cycle, and wherein said controller is adapted to find said relative stiffness from the pressure drops of said at least two different phases.

4. The system of claim 1, also comprising a flow sensor adapted to generate a signal indicative of flow in said body lumen, and wherein the controller is adapted to use the signal indicative of flow, obtained for the at least two flow conditions, to find an absolute stiffness of the body lumen from the pressure drops.

5. The system according to claim 1, also comprising a sleeve that surrounds the pressure-measuring catheter, the pressure-measuring catheter being adapted to withdraw into and extend out of the sleeve when it is inserted into the body lumen, wherein the controller is adapted to discern a distortion of the signal generated by the pressure gauge when the pressure-measuring catheter is withdrawn into the sleeve.

6. The system according to claim 1, also comprising a sleeve that surrounds the pressure-measuring catheter, the pressure-measuring catheter being adapted to withdraw into and extend out of the sleeve when it is inserted into the body lumen, wherein the controller is adapted to discern a distortion of the signal generated by the pressure gauge when the pressure-measuring catheter is withdrawn into the sleeve, wherein said sleeve includes a delivery catheter.

7. The system according to claim 1, also comprising a sleeve that surrounds the pressure-measuring catheter, the pressure-measuring catheter being adapted to withdraw into and extend out of the sleeve when it is inserted into the body lumen, wherein the controller is adapted to discern a distortion of the signal generated by the pressure gauge when the pressure-measuring catheter is withdrawn into the sleeve, wherein said sleeve includes a delivery catheter and wherein said controller is further configured to calculate a correction for said distortion to the signal and to apply said correction for said distortion to the signal generated by the pressure gauge when the pressure-measuring catheter is exposed to the flow in said body lumen, said correction based on a result of said discerning.

8. The system according to claim 1, also comprising a sleeve that surrounds the pressure-measuring catheter, the pressure-measuring catheter being adapted to withdraw into and extend out of the sleeve when it is inserted into the body lumen, wherein the controller is adapted to discern a distortion of the signal generated by the pressure gauge when the pressure-measuring catheter is withdrawn into the sleeve, wherein said distortion includes a common mode pressure distortion.

9. The system of claim 1, wherein a distance between said measuring locations is no more than 5 cm.

10. The system of claim 2, wherein each said opening of said first and second openings is substantially at a distal end of each respective said lumen of the catheter.

11. The system of claim 2, further comprising an interventional device for performing a treatment intervention on said body lumen and wherein said pressure-measuring catheter is used for at least one of verifying and monitoring said treatment intervention.

12. The system of claim 2, further comprising an interventional device for performing a treatment intervention on said body lumen and wherein said pressure-measuring catheter is used for at least one of verifying and monitoring said treatment intervention, and wherein said interventional device includes an ablation device.

13. The system of claim 1, wherein said controller is adapted to compute said pressure drop to an accuracy to within 0.1 mmHg.

14. A system according to claim 1, wherein said pressure gauge includes a differential pressure gauge.

15. A method of measuring stiffness of a blood vessel in a subject, the method comprising:
measuring a pressure drop across substantially the same portion of the blood vessel, for each of at least two different flow conditions; and
analyzing the measured pressure drops to determine a relative stiffness of the blood vessel.

16-17. (canceled)

18. A method according to claim 15, also comprising measuring the pressure drop for each of at least two flow conditions across at least another portion along the blood vessel, and analyzing the measured pressure drops to determine at least a relative stiffness comprises comparing the pressure drops measured across said same portion and said at least another portion to determine the relative stiffness, wherein said same portion and said at least another portion have one of the same length and different lengths.

19-20. (canceled)

21. A method according to claim 15, also comprising evaluating a reduction in blood flow caused by the resistance in the same portion based on the pressure drop.

22. (canceled)

23. A method according to claim 15, also comprising measuring a blood flow rate in the blood vessel at two different phases of a cardiac cycle, and analyzing the measured pressure drops comprises also using the measured blood flow rates and determining an absolute stiffness.

24-25. (canceled)

26. A method according to claim 15, also comprising using the determined stiffness to locate or evaluate one or more of a stenosis, a sclerotic lesion, and vulnerable plaque.

27-28. (canceled)

29. A method according claim 15, also comprising assessing an interventional treatment based on the determined stiffness, wherein said interventional treatment is of a renal denervation.

30-34. (canceled)

35. The system of claim 130, wherein said controller is adapted to compute a Fractional Flow Reserve indicator.

36-41. (canceled)

42. The method according to claim 15, wherein said measuring includes:
inserting a probe into a first region of fluid with a time-varying gauge pressure but negligible pressure drop between the sensing locations;
measuring an indicator of the time-varying gauge pressure in the first region;
sensing said pressure drop under the time-varying gauge pressure in the first region, and
inserting the catheter into a second region of fluid with a pressure drop between said sensing locations;
measuring the indicator of time-varying gauge pressure in the second region; and
sensing the pressure drop in the second region; and
said method further includes correcting a distortion of differential pressure between two sensing locations, comprising:
finding a restoration function of the indicator of time-varying gauge pressure in the first region for an output signal of said sensing in said first region; and
transforming an output signal of said sensing in the second region with the restoration function and a function of the measured indicator of time-varying gauge pressure to obtain a corrected pressure drop in the second region, wherein said transforming results in the corrected pressure that is accurate to within 0.05 mmHg.

43. The method according to claim 15, wherein said measuring includes:
inserting a probe into a first region of fluid with a time-varying gauge pressure but negligible pressure drop between the sensing locations;
measuring an indicator of the time-varying gauge pressure in the first region;
sensing said pressure drop under the time-varying gauge pressure in the first region, and
inserting the catheter into a second region of fluid with a pressure drop between said sensing locations;
measuring the indicator of time-varying gauge pressure in the second region,
sensing the pressure drop in the second region; and
said method further includes correcting a distortion of differential pressure between two sensing locations, comprising:
finding a restoration function of the indicator of lime-varying gauge pressure in the first region for an output signal of said sensing in said first region; and
transforming an output signal of said sensing in the second region with the restoration function and a function of the measured indicator of time-varying gauge pressure to obtain a corrected pressure drop in the second region;
wherein the restoration function transforms the indicator of gauge pressure to a linear combination of a finite number of terms selected from at least the one of the indicator, a first order time derivative of the indicator, a higher order derivative of the indicator, a linear function of the gauge pressure, a derivative of the liner function of the gauge pressure and a higher order derivative of the linear function of the gauge pressure.

44. (canceled)

45. The method according to claim 15, wherein said measuring includes:
inserting a probe into a first region of fluid with a time-varying gauge pressure but negligible pressure drop between the sensing locations;
measuring an indicator of the time-varying gauge pressure in the first region;
sensing said pressure drop under the time-varying gauge pressure in the first region, and
inserting the catheter into a second region of fluid with a pressure drop between said sensing locations;
measuring the indicator of time-varying gauge pressure in the second region,
sensing the pressure drop in the second region; and
said method further includes correcting a distortion of differential pressure between two sensing locations, comprising;
finding a restoration function of the indicator of time-varying gauge pressure in the first region for an output signal of said sensing in said first region; and
transforming an output signal of said sensing in the second region with the restoration function and a function of the measured indicator of time-varying gauge pressure to obtain a corrected pressure drop in the second region;
extending the probe out of the first to the second region wherein the second region is an interior of the body lumen, and the first region is a region with negligible flow.

46-49. (canceled)

50. A compound device for measuring a pressure drop in a body lumen in-vivo comprising:
a probe of a pressure drop between two sensing locations;
a sleeve that surrounds the pressure-measuring probe when it is inserted into the blood vessel, the pressure-measuring catheter being adapted to withdraw into and extend out of the sleeve;
a sensor generate a signal indicative the pressure drop between said sensing locations; and
a controller adapted to discern a distortion of said signal generated when the probe is surrounded by the sleeve.

51-53. (canceled)
